SEQUENCE LISTING (208 sequences)

SEQ ID NO: 1 - protein, 347 aa, Pseudomonas aeruginosa
PEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 2 - protein, 18 aa, Pseudomonas aeruginosa
ARGRIRNGALLRVYVPRS

SEQ ID NO: 3 - protein, 15 aa, Pseudomonas aeruginosa
RLVALYLAARLSWNQ

SEQ ID NO: 4 - protein, 15 aa, Pseudomonas aeruginosa
SQDLDAIWRGFYIAG

SEQ ID NO: 5 - protein, 15 aa, Pseudomonas aeruginosa
ARGRIRNGALLRVYV

SEQ ID NO: 6 - protein, 15 aa, Pseudomonas aeruginosa
RIRNGALLRVYVPRS

SEQ ID NO: 7 - protein, 15 aa, Artificial sequence
  Synthetic sequence: Peptide tested in I-MUNE assay
ASSSVSYMHWLQQKP

SEQ ID NO: 8 - protein, 15 aa, Artificial sequence
  Synthetic sequence: Peptide tested in I-MUNE assay
SVSYMHWLQQKPGTS

SEQ ID NO: 9 - protein, 15 aa, Artificial sequence
  Synthetic sequence: Peptide tested in I-MUNE assay
YMHWLQQKPGTSPKL

SEQ ID NO: 10 - protein, 15 aa, Artificial sequence
  Synthetic sequence: Peptide tested in I-MUNE assay
WLQQKPGTSPKLVIY

SEQ ID NO: 11 - protein, 15 aa, Artificial sequence
  Synthetic sequence: Peptide tested in I-MUNE assay
QKPGTSPKLVIYLTS

SEQ ID NO: 12 - protein, 15 aa, Artificial sequence
  Synthetic sequence: Peptide tested in I-MUNE assay
GTSPKLVIYLTSNLA

SEQ ID NO: 13 - protein, 15 aa, Artificial sequence
  Synthetic sequence: Peptide tested in I-MUNE assay
PKLVIYLTSNLASGV

SEQ ID NO: 14 - protein, 15 aa, Artificial sequence
  Synthetic sequence: Peptide tested in I-MUNE assay
VIYLTSNLASGVPAR

SEQ ID NO: 15 - protein, 15 aa, Artificial sequence
  Synthetic sequence: Peptide tested in I-MUNE assay
LTSNLASGVPARFSG

SEQ ID NO: 16 - protein, 15 aa, Artificial sequence
  Synthetic sequence: Peptide tested in I-MUNE assay
ASSSVSYMHWFQQKP

SEQ ID NO: 17 - protein, 15 aa, Artificial sequence
  Synthetic sequence: Peptide tested in I-MUNE assay
SVSYMHWFQQKPGTS

SEQ ID NO: 18 - protein, 15 aa, Artificial sequence
  Synthetic sequence: Peptide tested in I-MUNE assay
YMHWFQQKPGTSPKL

SEQ ID NO: 19 - protein, 15 aa, Artificial sequence
  Synthetic sequence: Peptide tested in I-MUNE assay
WFQQKPGTSPKLVIY

SEQ ID NO: 20 - protein, 15 aa, Artificial sequence
  Synthetic sequence: Peptide tested in I-MUNE assay
QKPGTSPKLVIYSTS

SEQ ID NO: 21 - protein, 15 aa, Artificial sequence
  Synthetic sequence: Peptide tested in I-MUNE assay
GTSPKLVIYSTSNLA

SEQ ID NO: 22 - protein, 15 aa, Artificial sequence
  Synthetic sequence: Peptide tested in I-MUNE assay
PKLVIYSTSNLASGV

SEQ ID NO: 23 - protein, 15 aa, Artificial sequence
  Synthetic sequence: Peptide tested in I-MUNE assay
VIYSTSNLASGVPAR

SEQ ID NO: 24 - protein, 15 aa, Artificial sequence
  Synthetic sequence: Peptide tested in I-MUNE assay
STSNLASGVPARFSG

SEQ ID NO: 25 - protein, 15 aa, Artificial sequence
  Synthetic sequence: Peptide tested in I-MUNE assay
MEVQLVESGGGLVKP

SEQ ID NO: 26 - protein, 15 aa, Artificial sequence
  Synthetic sequence: Peptide tested in I-MUNE assay
QLVESGGGLVKPGGS

SEQ ID NO: 27 - protein, 15 aa, Artificial sequence
  Synthetic sequence: Peptide tested in I-MUNE assay
ESGGGLVKPGGSLKL

SEQ ID NO: 28 - protein, 15 aa, Artificial sequence
  Synthetic sequence: Peptide tested in I-MUNE assay
GGLVKPGGSLKLSCA

SEQ ID NO: 29 - protein, 15 aa, Artificial sequence
  Synthetic sequence: Peptide tested in I-MUNE assay
VKPGGSLKLSCAASG

SEQ ID NO: 30 - protein, 15 aa, Artificial sequence
  Synthetic sequence: Peptide tested in I-MUNE assay
GGSLKLSCAASGFAF

SEQ ID NO: 31 - protein, 15 aa, Artificial sequence
  Synthetic sequence: Peptide tested in I-MUNE assay
LKLSCAASGFAFSIY

SEQ ID NO: 32 - protein, 15 aa, Artificial sequence
  Synthetic sequence: Peptide tested in I-MUNE assay
SCAASGFAFSIYDMS

SEQ ID NO: 33 - protein, 15 aa, Artificial sequence
  Synthetic sequence: Peptide tested in I-MUNE assay
ASGFAFSIYDMSWVR

SEQ ID NO: 34 - protein, 15 aa, Artificial sequence
  Synthetic sequence: Peptide tested in I-MUNE assay
FAFSIYDMSWVRQTP

SEQ ID NO: 35 - protein, 15 aa, Artificial sequence
  Synthetic sequence: Peptide tested in I-MUNE assay
SIYDMSWVRQTPEKC

SEQ ID NO: 36 - protein, 15 aa, Artificial sequence
  Synthetic sequence: Peptide tested in I-MUNE assay
DMSWVRQTPEKCLEW

SEQ ID NO: 37 - protein, 15 aa, Artificial sequence
  Synthetic sequence: Peptide tested in I-MUNE assay
WVRQTPEKCLEWVAY

SEQ ID NO: 38 - protein, 15 aa, Artificial sequence
  Synthetic sequence: Peptide tested in I-MUNE assay
QTPEKCLEWVAYISS

SEQ ID NO: 39 - protein, 15 aa, Artificial sequence
  Synthetic sequence: Peptide tested in I-MUNE assay
EKCLEWVAYISSGGG

SEQ ID NO: 40 - protein, 15 aa, Artificial sequence
  Synthetic sequence: Peptide tested in I-MUNE assay
LEWVAYISSGGGTTY

SEQ ID NO: 41 - protein, 15 aa, Artificial sequence
  Synthetic sequence: Peptide tested in I-MUNE assay
VAYISSGGGTTYYPD

SEQ ID NO: 42 - protein, 15 aa, Artificial sequence
  Synthetic sequence: Peptide tested in I-MUNE assay
ISSGGGTTYYPDTVK

SEQ ID NO: 43 - protein, 15 aa, Artificial sequence
  Synthetic sequence: Peptide tested in I-MUNE assay
GGGTTYYPDTVKGRF

SEQ ID NO: 44 - protein, 15 aa, Artificial sequence
  Synthetic sequence: Peptide tested in I-MUNE assay
TTYYPDTVKGRFTIS

SEQ ID NO: 45 - protein, 15 aa, Artificial sequence
  Synthetic sequence: Peptide tested in I-MUNE assay
YPDTVKGRFTISRDN

SEQ ID NO: 46 - protein, 15 aa, Artificial sequence
  Synthetic sequence: Peptide tested in I-MUNE assay
TVKGRFTISRDNAKN

SEQ ID NO: 47 - protein, 15 aa, Artificial sequence
  Synthetic sequence: Peptide tested in I-MUNE assay
GRFTISRDNAKNTLY

SEQ ID NO: 48 - protein, 15 aa, Artificial sequence
  Synthetic sequence: Peptide tested in I-MUNE assay
TISRDNAKNTLYLQM

SEQ ID NO: 49 - protein, 15 aa, Artificial sequence
  Synthetic sequence: Peptide tested in I-MUNE assay
RDNAKNTLYLQMSSL

SEQ ID NO: 50 - protein, 15 aa, Artificial sequence
  Synthetic sequence: Peptide tested in I-MUNE assay
AKNTLYLQMSSLKSE

SEQ ID NO: 51 - protein, 15 aa, Artificial sequence
  Synthetic sequence: Peptide tested in I-MUNE assay
TLYLQMSSLKSEDTA

SEQ ID NO: 52 - protein, 15 aa, Artificial sequence
  Synthetic sequence: Peptide tested in I-MUNE assay
LQMSSLKSEDTAMYY

SEQ ID NO: 53 - protein, 15 aa, Artificial sequence
  Synthetic sequence: Peptide tested in I-MUNE assay
SSLKSEDTAMYYCAR

SEQ ID NO: 54 - protein, 15 aa, Artificial sequence
  Synthetic sequence: Peptide tested in I-MUNE assay
KSEDTAMYYCARHSG

SEQ ID NO: 55 - protein, 15 aa, Artificial sequence
  Synthetic sequence: Peptide tested in I-MUNE assay
DTAMYYCARHSGYGT

SEQ ID NO: 56 - protein, 15 aa, Artificial sequence
  Synthetic sequence: Peptide tested in I-MUNE assay
MYYCARHSGYGTHWG

SEQ ID NO: 57 - protein, 15 aa, Artificial sequence
  Synthetic sequence: Peptide tested in I-MUNE assay
CARHSGYGTHWGVLF

SEQ ID NO: 58 - protein, 15 aa, Artificial sequence
  Synthetic sequence: Peptide tested in I-MUNE assay
HSGYGTHWGVLFAYW

SEQ ID NO: 59 - protein, 15 aa, Artificial sequence
  Synthetic sequence: Peptide tested in I-MUNE assay
YGTHWGVLFAYWGQG

SEQ ID NO: 60 - protein, 15 aa, Artificial sequence
  Synthetic sequence: Peptide tested in I-MUNE assay
HWGVLFAYWGQGTLV

SEQ ID NO: 61 - protein, 15 aa, Artificial sequence
  Synthetic sequence: Peptide tested in I-MUNE assay
VLFAYWGQGTLVTVS

SEQ ID NO: 62 - protein, 15 aa, Artificial sequence
  Synthetic sequence: Peptide tested in I-MUNE assay
AYWGQGTLVTVSAKA

SEQ ID NO: 63 - protein, 15 aa, Artificial sequence
  Synthetic sequence: Peptide tested in I-MUNE assay
GQGTLVTVSAKASGG

SEQ ID NO: 64 - protein, 15 aa, Artificial sequence
  Synthetic sequence: Peptide tested in I-MUNE assay
TLVTVSAKASGGPEG

SEQ ID NO: 65 - protein, 15 aa, Artificial sequence
  Synthetic sequence: Peptide tested in I-MUNE assay
TVSAKASGGPEGGSL

SEQ ID NO: 66 - protein, 15 aa, Artificial sequence
  Synthetic sequence: Peptide tested in I-MUNE assay
AKASGGPEGGSLAAL

SEQ ID NO: 67 - protein, 15 aa, Artificial sequence
  Synthetic sequence: Peptide tested in I-MUNE assay
SGGPEGGSLAALTAH

SEQ ID NO: 68 - protein, 15 aa, Pseudomonas aeruginosa
PEGGSLAALTAHQAC

SEQ ID NO: 69 - protein, 15 aa, Pseudomonas aeruginosa
GSLAALTAHQACHLP

SEQ ID NO: 70 - protein, 15 aa, Pseudomonas aeruginosa
AALTAHQACHLPLET

SEQ ID NO: 71 - protein, 15 aa, Pseudomonas aeruginosa
TAHQACHLPLETFTR

SEQ ID NO: 72 - protein, 15 aa, Pseudomonas aeruginosa
QACHLPLETFTRHRQ

SEQ ID NO: 73 - protein, 15 aa, Pseudomonas aeruginosa
HLPLETFTRHRQPRG

SEQ ID NO: 74 - protein, 15 aa, Pseudomonas aeruginosa
LETFTRHRQPRGWEQ

SEQ ID NO: 75 - protein, 15 aa, Pseudomonas aeruginosa
FTRHRQPRGWEQLEQ

SEQ ID NO: 76 - protein, 15 aa, Pseudomonas aeruginosa
HRQPRGWEQLEQCGY

SEQ ID NO: 77 - protein, 15 aa, Pseudomonas aeruginosa
PRGWEQLEQCGYPVQ

SEQ ID NO: 78 - protein, 15 aa, Pseudomonas aeruginosa
WEQLEQCGYPVQRLV

SEQ ID NO: 79 - protein, 15 aa, Pseudomonas aeruginosa
LEQCGYPVQRLVALY

SEQ ID NO: 80 - protein, 15 aa, Pseudomonas aeruginosa
CGYPVQRLVALYLAA

SEQ ID NO: 81 - protein, 15 aa, Pseudomonas aeruginosa
PVQRLVALYLAARLS

SEQ ID NO: 82 - skipped

SEQ ID NO: 83 - protein, 15 aa, Pseudomonas aeruginosa
ALYLAARLSWNQVDQ

SEQ ID NO: 84 - protein, 15 aa, Pseudomonas aeruginosa
LAARLSWNQVDQVIR

SEQ ID NO: 85 - protein, 15 aa, Pseudomonas aeruginosa
RLSWNQVDQVIRNAL

SEQ ID NO: 86 - protein, 15 aa, Pseudomonas aeruginosa
WNQVDQVIRNALASP

SEQ ID NO: 87 - protein, 15 aa, Pseudomonas aeruginosa
VDQVIRNALASPGSG

SEQ ID NO: 88 - protein, 15 aa, Pseudomonas aeruginosa
VIRNALASPGSGGDL

SEQ ID NO: 89 - protein, 15 aa, Pseudomonas aeruginosa
NALASPGSGGDLGEA

SEQ ID NO: 90 - protein, 15 aa, Pseudomonas aeruginosa
ASPGSGGDLGEAIRE

SEQ ID NO: 91 - protein, 15 aa, Pseudomonas aeruginosa
GSGGDLGEAIREQPE

SEQ ID NO: 92 - protein, 15 aa, Pseudomonas aeruginosa
GDLGEAIREQPEQAR

SEQ ID NO: 93 - protein, 15 aa, Pseudomonas aeruginosa
GEAIREQPEQARLAL

SEQ ID NO: 94 - protein, 15 aa, Pseudomonas aeruginosa
IREQPEQARLALTLA

SEQ ID NO: 95 - protein, 15 aa, Pseudomonas aeruginosa
QPEQARLALTLAAAE

SEQ ID NO: 96 - protein, 15 aa, Pseudomonas aeruginosa
QARLALTLAAAESER

SEQ ID NO: 97 - protein, 15 aa, Pseudomonas aeruginosa
LALTLAAAESERFVR

SEQ ID NO: 98 - protein, 15 aa, Pseudomonas aeruginosa
TLAAAESERFVRQGT

SEQ ID NO: 99 - protein, 15 aa, Pseudomonas aeruginosa
AAESERFVRQGTGND

SEQ ID NO: 100 - protein, 15 aa, Pseudomonas aeruginosa
SERFVRQGTGNDEAG

SEQ ID NO: 101 - protein, 15 aa, Pseudomonas aeruginosa
FVRQGTGNDEAGAAN

SEQ ID NO: 102 - protein, 15 aa, Pseudomonas aeruginosa
QGTGNDEAGAANGPA

SEQ ID NO: 103 - protein, 15 aa, Pseudomonas aeruginosa
GNDEAGAANGPADSG

SEQ ID NO: 104 - protein, 15 aa, Pseudomonas aeruginosa
EAGAANGPADSGDAL

SEQ ID NO: 105 - protein, 15 aa, Pseudomonas aeruginosa
AANGPADSGDALLER

SEQ ID NO: 106 - protein, 15 aa, Pseudomonas aeruginosa
GPADSGDALLERNYP

SEQ ID NO: 107 - protein, 15 aa, Pseudomonas aeruginosa
DSGDALLERNYPTGA

SEQ ID NO: 108 - protein, 15 aa, Pseudomonas aeruginosa
DALLERNYPTGAEFL

SEQ ID NO: 109 - protein, 15 aa, Pseudomonas aeruginosa
LERNYPTGAEFLGDG

SEQ ID NO: 110 - protein, 15 aa, Pseudomonas aeruginosa
NYPTGAEFLGDGGDV

SEQ ID NO: 111 - protein, 15 aa, Pseudomonas aeruginosa
TGAEFLGDGGDVSFS

SEQ ID NO: 112 - protein, 15 aa, Pseudomonas aeruginosa
EFLGDGGDVSFSTRG

SEQ ID NO: 113 - protein, 15 aa, Pseudomonas aeruginosa
GDGGDVSFSTRGTQN

SEQ ID NO: 114 - protein, 15 aa, Pseudomonas aeruginosa
GDVSFSTRGTQNWTV

SEQ ID NO: 115 - protein, 15 aa, Pseudomonas aeruginosa
SFSTRGTQNWTVERL

SEQ ID NO: 116 - protein, 15 aa, Pseudomonas aeruginosa
TRGTQNWTVERLLQA

SEQ ID NO: 117 - protein, 15 aa, Pseudomonas aeruginosa
TQNWTVERLLQAHRQ

SEQ ID NO: 118 - protein, 15 aa, Pseudomonas aeruginosa
WTVERLLQAHRQLEE

SEQ ID NO: 119 - protein, 15 aa, Pseudomonas aeruginosa
ERLLQAHRQLEERGY

SEQ ID NO: 120 - protein, 15 aa, Pseudomonas aeruginosa
LQAHRQLEERGYVFV

SEQ ID NO: 121 - protein, 15 aa, Pseudomonas aeruginosa
HRQLEERGYVFVGYH

SEQ ID NO: 122 - protein, 15 aa, Pseudomonas aeruginosa
LEERGYVFVGYHGTF

SEQ ID NO: 123 - protein, 15 aa, Pseudomonas aeruginosa
RGYVFVGYHGTFLEA

SEQ ID NO: 124 - protein, 15 aa, Pseudomonas aeruginosa
VFVGYHGTFLEAAQS

SEQ ID NO: 125 - protein, 15 aa, Pseudomonas aeruginosa
GYHGTFLEAAQSIVF

SEQ ID NO: 126 - protein, 15 aa, Pseudomonas aeruginosa
GTFLEAAQSIVFGGV

SEQ ID NO: 127 - protein, 15 aa, Pseudomonas aeruginosa
LEAAQSIVFGGVRAR

SEQ ID NO: 128 - protein, 15 aa, Pseudomonas aeruginosa
AQSIVFGGVRARSQD

SEQ ID NO: 129 - protein, 15 aa, Pseudomonas aeruginosa
IVFGGVRARSQDLDA

SEQ ID NO: 130 - protein, 15 aa, Pseudomonas aeruginosa
GGVRARSQDLDAIWR

SEQ ID NO: 131 - protein, 15 aa, Pseudomonas aeruginosa
RARSQDLDAIWRGFY

SEQ ID NO: 132 - protein, 15 aa, Pseudomonas aeruginosa
LDAIWRGFYIAGDPA

SEQ ID NO: 133 - protein, 15 aa, Pseudomonas aeruginosa
IWRGFYIAGDPALAY

SEQ ID NO: 134 - protein, 15 aa, Pseudomonas aeruginosa
GFYIAGDPALAYGYA

SEQ ID NO: 135 - protein, 15 aa, Pseudomonas aeruginosa
IAGDPALAYGYAQDQ

SEQ ID NO: 136 - protein, 15 aa, Pseudomonas aeruginosa
DPALAYGYAQDQEPD

SEQ ID NO: 137 - protein, 15 aa, Pseudomonas aeruginosa
LAYGYAQDQEPDARG

SEQ ID NO: 138 - protein, 15 aa, Pseudomonas aeruginosa
GYAQDQEPDARGRIR

SEQ ID NO: 139 - protein, 15 aa, Pseudomonas aeruginosa
QDQEPDARGRIRNGA

SEQ ID NO: 140 - protein, 15 aa, Pseudomonas aeruginosa
EPDARGRIRNGALLR

SEQ ID NO: 141 - protein, 15 aa, Pseudomonas aeruginosa
NGALLRVYVPRSSLP

SEQ ID NO: 142 - protein, 15 aa, Pseudomonas aeruginosa
LLRVYVPRSSLPGFY

SEQ ID NO: 143 - protein, 15 aa, Pseudomonas aeruginosa
VYVPRSSLPGFYRTS

SEQ ID NO: 144 - protein, 15 aa, Pseudomonas aeruginosa
PRSSLPGFYRTSLTL

SEQ ID NO: 145 - protein, 15 aa, Pseudomonas aeruginosa
SLPGFYRTSLTLAAP

SEQ ID NO: 146 - protein, 15 aa, Pseudomonas aeruginosa
GFYRTSLTLAAPEAA

SEQ ID NO: 147 - protein, 15 aa, Pseudomonas aeruginosa
RTSLTLAAPEAAGEV

SEQ ID NO: 148 - protein, 15 aa, Pseudomonas aeruginosa
LTLAAPEAAGEVERL

SEQ ID NO: 149 - protein, 15 aa, Pseudomonas aeruginosa
AAPEAAGEVERLIGH

SEQ ID NO: 150 - protein, 15 aa, Pseudomonas aeruginosa
EAAGEVERLIGHPLP

SEQ ID NO: 151 - protein, 15 aa, Pseudomonas aeruginosa
GEVERLIGHPLPLRL

SEQ ID NO: 152 - protein, 15 aa, Pseudomonas aeruginosa
ERLIGHPLPLRLDAI

SEQ ID NO: 153 - protein, 15 aa, Pseudomonas aeruginosa
IGHPLPLRLDAITGP

SEQ ID NO: 154 - protein, 15 aa, Pseudomonas aeruginosa
PLPLRLDAITGPEEE

SEQ ID NO: 155 - protein, 15 aa, Pseudomonas aeruginosa
LRLDAITGPEEEGGR

SEQ ID NO: 156 - protein, 15 aa, Pseudomonas aeruginosa
DAITGPEEEGGRLET

SEQ ID NO: 157 - protein, 15 aa, Pseudomonas aeruginosa
TGPEEEGGRLETILG

SEQ ID NO: 158 - protein, 15 aa, Pseudomonas aeruginosa
EEEGGRLETILGWPL

SEQ ID NO: 159 - protein, 15 aa, Pseudomonas aeruginosa
GGRLETILGWPLAER

SEQ ID NO: 160 - protein, 15 aa, Pseudomonas aeruginosa
LETILGWPLAERTVV

SEQ ID NO: 161 - protein, 15 aa, Pseudomonas aeruginosa
ILGWPLAERTVVIPS

SEQ ID NO: 162 - protein, 15 aa, Pseudomonas aeruginosa
WPLAERTVVIPSAIP

SEQ ID NO: 163 - protein, 15 aa, Pseudomonas aeruginosa
AERTVVIPSAIPTDP

SEQ ID NO: 164 - protein, 15 aa, Pseudomonas aeruginosa
TVVIPSAIPTDPRNV

SEQ ID NO: 165 - protein, 15 aa, Pseudomonas aeruginosa
IPSAIPTDPRNVGGD

SEQ ID NO: 166 - protein, 15 aa, Pseudomonas aeruginosa
AIPTDPRNVGGDLDP

SEQ ID NO: 167 - protein, 15 aa, Pseudomonas aeruginosa
TDPRNVGGDLDPSSI

SEQ ID NO: 168 - protein, 15 aa, Pseudomonas aeruginosa
RNVGGDLDPSSIPDK

SEQ ID NO: 169 - protein, 15 aa, Pseudomonas aeruginosa
GGDLDPSSIPDKEQA

SEQ ID NO: 170 - protein, 15 aa, Pseudomonas aeruginosa
LDPSSIPDKEQAISA

SEQ ID NO: 171 - protein, 15 aa, Pseudomonas aeruginosa
SSIPDKEQAISALPD

SEQ ID NO: 172 - protein, 15 aa, Pseudomonas aeruginosa
PDKEQAISALPDYAS

SEQ ID NO: 173 - protein, 15 aa, Pseudomonas aeruginosa
EQAISALPDYASQPG

SEQ ID NO: 174 - protein, 15 aa, Pseudomonas aeruginosa
ISALPDYASQPGKPP

SEQ ID NO: 175 - protein, 15 aa, Pseudomonas aeruginosa
LPDYASQPGKPPRED

SEQ ID NO: 176 - protein, 15 aa, Pseudomonas aeruginosa
DYASQPGKPPREDLK

SEQ ID NO: 177 - protein, 15 aa, Pseudomonas aeruginosa
MDIQMTQTTSSLSAS

SEQ ID NO: 178 - protein, 15 aa, Artificial sequence
  Synthetic sequence: Peptide tested in I-MUNE assay
QMTQTTSSLSASLGD

SEQ ID NO: 179 - protein, 15 aa, Artificial sequence
  Synthetic sequence: Peptide tested in I-MUNE assay
QTTSSLSASLGDRVT

SEQ ID NO: 180 - protein, 15 aa, Artificial sequence
  Synthetic sequence: Peptide tested in I-MUNE assay
SSLSASLGDRVTISC

SEQ ID NO: 181 - protein, 15 aa, Artificial sequence
  Synthetic sequence: Peptide tested in I-MUNE assay
SASLGDRVTISCRAS

SEQ ID NO: 182 - protein, 15 aa, Artificial sequence
  Synthetic sequence: Peptide tested in I-MUNE assay
LGDRVTISCRASQDI

SEQ ID NO: 183 - protein, 15 aa, Artificial sequence
  Synthetic sequence: Peptide tested in I-MUNE assay
RVTISCRASQDISNY

SEQ ID NO: 184 - protein, 15 aa, Artificial sequence
  Synthetic sequence: Peptide tested in I-MUNE assay
ISCRASQDISNYLNW

SEQ ID NO: 185 - protein, 15 aa, Artificial sequence
  Synthetic sequence: Peptide tested in I-MUNE assay
RASQDISNYLNWYQQ

SEQ ID NO: 186 - protein, 15 aa, Artificial sequence
  Synthetic sequence: Peptide tested in I-MUNE assay
QDISNYLNWYQQKPD

SEQ ID NO: 187 - protein, 15 aa, Artificial sequence
  Synthetic sequence: Peptide tested in I-MUNE assay
SNYLNWYQQKPDGTV

SEQ ID NO: 188 - protein, 15 aa, Artificial sequence
  Synthetic sequence: Peptide tested in I-MUNE assay
LNWYQQKPDGTVKLL

SEQ ID NO: 189 - protein, 15 aa, Artificial sequence
  Synthetic sequence: Peptide tested in I-MUNE assay
YQQKPDGTVKLLIYY

SEQ ID NO: 190 - protein, 15 aa, Artificial sequence
  Synthetic sequence: Peptide tested in I-MUNE assay
KPDGTVKLLIYYTSI

SEQ ID NO: 191 - protein, 15 aa, Artificial sequence
  Synthetic sequence: Peptide tested in I-MUNE assay
GTVKLLIYYTSILHS

SEQ ID NO: 192 - protein, 15 aa, Artificial sequence
  Synthetic sequence: Peptide tested in I-MUNE assay
KLLIYYTSILHSGVP

SEQ ID NO: 193 - protein, 15 aa, Artificial sequence
  Synthetic sequence: Peptide tested in I-MUNE assay
IYYTSILHSGVPSRF

SEQ ID NO: 194 - protein, 15 aa, Artificial sequence
  Synthetic sequence: Peptide tested in I-MUNE assay
TSILHSGVPSRFSGS

SEQ ID NO: 195 - protein, 15 aa, Artificial sequence
  Synthetic sequence: Peptide tested in I-MUNE assay
LHSGVPSRFSGSGSG

SEQ ID NO: 196 - protein, 15 aa, Artificial sequence
  Synthetic sequence: Peptide tested in I-MUNE assay
GVPSRFSGSGSGTDY

SEQ ID NO: 197 - protein, 15 aa, Artificial sequence
  Synthetic sequence: Peptide tested in I-MUNE assay
SRFSGSGSGTDYSLT

SEQ ID NO: 198 - protein, 15 aa, Artificial sequence
  Synthetic sequence: Peptide tested in I-MUNE assay
SGSGSGTDYSLTISN

SEQ ID NO: 199 - protein, 15 aa, Artificial sequence
  Synthetic sequence: Peptide tested in I-MUNE assay
GSGTDYSLTISNLEQ

SEQ ID NO: 200 - protein, 15 aa, Artificial sequence
  Synthetic sequence: Peptide tested in I-MUNE assay
TDYSLTISNLEQEDF

SEQ ID NO: 201 - protein, 15 aa, Artificial sequence
  Synthetic sequence: Peptide tested in I-MUNE assay
SLTISNLEQEDFATY

SEQ ID NO: 202 - protein, 15 aa, Artificial sequence
  Synthetic sequence: Peptide tested in I-MUNE assay
ISNLEQEDFATYFCQ

SEQ ID NO: 203 - protein, 15 aa, Artificial sequence
  Synthetic sequence: Peptide tested in I-MUNE assay
LEQEDFATYFCQQGN

SEQ ID NO: 204 - protein, 15 aa, Artificial sequence
  Synthetic sequence: Peptide tested in I-MUNE assay
EDFATYFCQQGNTLP

SEQ ID NO: 205 - protein, 15 aa, Artificial sequence
  Synthetic sequence: Peptide tested in I-MUNE assay
ATYFCQQGNTLPWTF

SEQ ID NO: 206 - protein, 15 aa, Artificial sequence
  Synthetic sequence: Peptide tested in I-MUNE assay
FCQQGNTLPWTFGCG

SEQ ID NO: 207 - protein, 15 aa, Artificial sequence
  Synthetic sequence: Peptide tested in I-MUNE assay
QGNTLPWTFGCGTKL

SEQ ID NO: 208 - protein, 15 aa, Artificial sequence
  Synthetic sequence: Peptide tested in I-MUNE assay
TLPWTFGCGTKLEIK

(19) United States
(12) Patent Application Publication
(10) Pub. No.: US 2008/0193976 A1
(43) Pub. Date: Aug. 14, 2008

(54) Pseudomonas Exotoxin A Cd4+T-Cell Epitopes

(76) Inventor: Fiona A. Harding, Santa Clara, CA (US)

Correspondence Address: MEDIMMUNE, LLC, ONE MEDIMMUNE WAY, GAITHERSBURG, MD, 20878, US

(21) Appl. No.: 12/066,709

(22) PCT Filed: Sep. 12, 2006
(86) PCT No.: PCT/GB2006/003384

§ 371 Date: Mar. 13, 2008

Related U.S. Application Data
(60) Provisional application No. 60/716,862, filed on Sep. 14, 2005.

(51) Int. Cl. C12P 21/00; C07K 7/08; C07K 14/21; C07K 16/46; C07H 21/04; C12N 15/63; C12N 5/06

(52) U.S. Cl. 435/69.1; 530/326; 530/350; 530/391.7; 530/391.9; 536/23.7; 435/320.1; 435/325

(57) ABSTRACT

The present invention provides PE CD4+T-cell epitopes, as well as novel variants that exhibit reduced immunogenic responses, as compared to the parental PE. The present invention further provides DNA molecules that encode novel PE variants, host cells comprising DNA encoding novel PE variants, as well as methods for making PEs less immunogenic. In addition, the present invention provides various compositions that comprise these PE variants that are less immunogenic than wild-type PEs.

### FIELD OF THE INVENTION

The present invention provides Pseudomonas exotoxin A (PE) CD4+T-cell epitopes, as well as novel variants that exhibit reduced immunogenic responses, as compared to the parental Pseudomonas exotoxin A. The present invention further provides DNA molecules that encode novel Pseudomonas exotoxin A variants, and host cells comprising DNA encoding novel PE variants, as well as methods for making Pseudomonas exotoxin A less immunogenic. In addition, the present invention provides various compositions that comprise these Pseudomonas exotoxin A variants that are less immunogenic than the wild-type Pseudomonas exotoxin A. In some specific embodiments, the present invention provides Pseudomonas exotoxin A variants with reduced immunogenicity that are identified and/or characterized using the methods of the present invention.

### BACKGROUND OF THE INVENTION

Pseudomonas exotoxin A is an enzyme produced by P. aeruginosa. It is lethal for some non-human animals and may play a role in human disease, as patients that are bacteremic with exotoxin A-producing Pseudomonas strains have a higher mortality rate than patients with non-exotoxin A producing organisms. The toxin is a single polypeptide chain comprised of 613 amino acids. Crystallographic studies and mutational analyses of the toxin have shown that consists of three domains with distinctive functions (See e.g., Chaudhary et al., Proc. Natl. Acad. Sci. USA 87:308-312 [1990]; and Pastan et al., Meth. Mol. Biol., 248:503-518 [2003]). There is an amino-terminal domain cell receptor-binding domain (domain I), a middle translocation domain (domain II), and a carboxy-terminal activity domain (domain III). Domain III acts by catalyzing ADP-ribosylation and inactivating elongation factor 2. This inhibits protein synthesis within cells and leads to cell death.

Recently, PE has found use in recombinant immunotoxins. In most cases, these chimeric proteins are comprised of the Fv portion of a monoclonal antibody fused to a portion of PE. The Fv replaces the cell-binding domain of the toxin, serving to direct the toxin to cancer cells that express a target antigen. Because of their potency and ability to kill cells that are resistant to standard chemotherapy, recombinant immunotoxins are very attractive agents for cancer treatment. Indeed, these complicated molecules have shown some promise in cancer therapy trials. However, as with all protein-based therapeutics, there are concerns about the immunogenicity of the immunotoxins themselves. Thus, there remains a need in the art to generate and use PE with reduced immunogenicity.

### SUMMARY OF THE INVENTION

The present invention provides Pseudomonas exotoxin A (PE) CD4+T-cell epitopes, as well as novel variants that exhibit reduced immunogenic responses, as compared to the parental Pseudomonas exotoxin A. The present invention further provides DNA molecules that encode novel PE variants, and host cells comprising DNA encoding novel PE variants, as well as methods for making PEs less immunogenic. In addition, the present invention provides various compositions that comprise these PE variants that are less immunogenic than the wild-type PEs. In some specific embodiments, the present invention provides PE variants with reduced immunogenicity that are identified and/or characterized using the methods of the present invention.

The present invention provides methods for identifying at least one T-cell epitope of PE, comprising the steps of: (a) obtaining from a single human blood source, a solution of dendritic cells and a solution of naive CD4+ and/or CD8+T-cells; (b) differentiating the dendritic cells to produce a solution of differentiated dendritic cells; (c) combining the solution of differentiated dendritic cells and the naive CD4+ and/or CD8+T-cells with peptide fragments of the PE; and (d) measuring proliferation of the T-cells in the step (c). In some alternative embodiments, the PE comprises at least a portion of the sequence set forth in SEQ ID NO:1. The present invention further provides epitopes identified using the above-described method.

The present invention also provides methods for reducing the immunogenicity of PE comprising the steps of: (a) identifying at least one T-cell epitope in the PE by (i) contacting an adherent monocyte-derived dendritic cell that has been differentiated by exposure to at least one cytokine in vitro, with at least one peptide comprising the T-cell epitope; and (ii) contacting the dendritic cell and the peptide with a naive T-cell, wherein the naive T-cell has been obtained from the same source as the adherent monocyte-derived dendritic cell, and whereby the T-cell proliferates in response to the peptide; and (b) modifying the PE to neutralize the T-cell epitope to produce a variant PE, such that the variant PE induces less than or substantially equal to the baseline proliferation of the naive T-cells. In some alternative embodiments, the PE comprises at least a portion of the sequence set forth in SEQ ID NO:1. In some embodiments, the present invention provides PE variants produced using the above-described method for production of PEs having reduced immunogenicity.

In some embodiments, the epitope of the PE is modified by: (a) substituting the amino acid sequence of the T-cell epitope with an analogous sequence from a homolog of the PE, wherein the substitution substantially mimics the major tertiary structure attributes of the T-cell epitope. In some preferred embodiments, the PE is modified by altering at least one epitope selected from the group consisting of SEQ ID NOS: 2, 3, 4, 5, and 6. In some embodiments, the epitope is modified by substituting an amino acid sequence for a residue corresponding to at least one of the epitopes, while in other embodiments, the epitope is modified by deleting an amino acid sequence for a residue corresponding to at least one of the epitopes, and in still further embodiments, the epitope is modified by adding an amino acid to at least one of the epitopes. The present invention further provides PEs produced using the above method.

The present invention also provides variant PEs comprising at least one alteration in at least one epitope comprising an amino acid sequence. In some particularly preferred embodiments, the immunogenic response produced by the variant PE is less than the immunogenic response produced by wild-type PE. However, in some other embodiments, the immunogenic response produced by the variant is greater than the immunogenic response produced by wild-type PE.

The present invention further provides compositions comprising nucleic acids sequences encoding variant PEs, as well as expression vectors that comprise the nucleic acid, and host cells transformed with the expression vectors.

The present invention still further provides pharmaceutical and consumer-related products, including, but not limited to such compositions as compositions that comprise the variant PEs of the present invention. In some embodiments, the PE variants find use in compositions such as those used to treat abnormal cells (e.g., cancer cells). However, it is also contemplated that the present invention will find use in targeted therapy methods that do not rely upon the use of antibodies and/or antibody components. Indeed, the present invention provides variant PEs suitable for use in various applications, settings, and compositions.

### DESCRIPTION OF THE INVENTION

The present invention provides PE CD4+T-cell epitopes, as well as novel variants that exhibit reduced immunogenic responses, as compared to the parental PE. The present invention further provides DNA molecules that encode novel PE variants, and host cells comprising DNA encoding novel PE variants, as well as methods for making PEs less immunogenic. In addition, the present invention provides various compositions that comprise these PE variants that are less immunogenic than the wild-type PEs. In some specific embodiments, the present invention provides PE peptides and variants with reduced immunogenicity identified and/or characterized using the methods of the present invention.

The “PE38” exotoxin is a recombinant variant of Pseudomonas exotoxin A (See e.g., Roscoe et al., Eur. J. Immunol., 27:1459-1468 [1997]). It has been modified to reduce its inherent toxicity by removing the cell-binding domain of the molecule. This truncated toxin molecule is highly effective at killing cells, but only when the molecule is internalized. Immunotoxin constructs containing the PE38 toxin coupled to either intact antibodies or to antibody fragments have been created. The antibody or antibody fragment confers specificity to the toxin, and binding to cell surface structures that undergo internalization provide a mechanism whereby the toxin can enter the cell and thereby kill it. This strategy has been remarkable effective for hematological malignancies such as hairy cell leukemia (HCL) (See e.g., Kreitman et al., J. Clin. Oncol., 18:1622-1636 [2000]).

In contrast to the success with HCL, immunotoxin strategy has been less effective in treating other forms of cancer. For example, it was found that for patients carrying solid tumors that a profound neutralizing immune response to the toxin molecule limited the efficacy of a PE38 immunotoxin construct (See e.g., Kreitman et al., J. Clin. Oncol., supra). Although it is not intended that the present invention be limited to any particular mechanism, it is contemplated that a likely reason for the difference between the success with hematological tumors versus solid tumors is that the target for HCL is a B-cell antigen. Thus, the immunotoxin immunosuppressed treated patients, whereas the solid tumor-possessing patients were largely immuno-intact. The present invention provides the means to expand the usage of immunotoxins in both hematological cancer patients and into other disease areas, as it is contemplated that a less immunogenic version of the toxin will find use in these applications.

As described in greater detail herein, an epitope map of the protein toxin PE38 was generated using the I-MUNE® assay system described below. One major epitope and two minor epitopes were identified. The overall background rate within the dataset indicates immunologic pre-exposure to the protein in the donor population. The I-MUNE® assay data donors were also HLA typed for HLA-DR and DQ. Associations between the presence of particular HLA class II alleles and response to all three prominent regions were also assessed.

### DEFINITIONS

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, N.Y. (1991) provide those of skill in the art with a general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular “a”, “an” and “the” includes the plural reference unless the context clearly indicates otherwise.

As used herein, “PE” refers to the enzyme commonly known as Pseudomonas exotoxin A. In some preferred embodiments, the PE of the present invention is native enzyme, while in some other preferred embodiments, the PE of the present invention is recombinant. In some further preferred embodiments, the PE is “PE38,” a 38 kDa derivative of the 66 kDa Pseudomonas exotoxin PE, in which the cell-binding domain of PE (amino acids 1-252) and amino acids 365-380 have been deleted (See e.g., Roscoe et al., supra). In some additionally preferred embodiments, the PEs of the present invention are modified, such that the PEs stimulate a lower immunogenic reaction than native PEs.

Some PE mutants are disclosed in WO2005/052006 and Bang et al. Clinical Cancer Research 1545 Vol. 11, 1545-1550, Feb. 15, 2005. Such mutants, e.g. wherein the PE has a glycine, alanine, valine, leucine, or isoleucine in place of arginine at the position corresponding to position 490 of mature PE (at 505 in GenBank entry AAB59097 of 11 Feb. 2002 since that includes the 25 amino acid leader sequence), which is position 224 of PE38 of SEQ ID NO: 1 herein, especially in which alanine is substituted for arginine at position 490 (R490A), may be subject to the present invention. The present invention allows for alteration of any parental PE, whether wild-type or mutant, bearing in mind that different mutations may have various, potentially independent effects on PE activity, toxicity or other property. For instance, according to WO2005/052006 the R490A mutation can double the toxicity of PE, and this mutation can easily be engineered into the various forms of modified PEs previously developed in the art (such as PE40, PE38, PE37, PE35, PE4E, PE38QQR, and PE38KDEL) to increase their potency and activity. It is expected that this will permit reducing the dose of PE-based immunotoxins required to produce a desired clinical result, which should reduce the possibility of undesirable side effects. Conversely, the same dose of PE-based immunotoxin can be administered, but with more potent effect. The present invention is concerned with altering a PE, e.g. PE38, to achieve a different effect, namely a reduced immunogenic response, and may be applied to wild-type PE or any other parental PE molecule, e.g. a PE with an alteration at position 490 or the corresponding residue, e.g. PE38 of SEQ ID NO: 1 herein with an alteration at position 224.

As used herein, the term “recombinant oligonucleotide” refers to an oligonucleotide created using molecular biological manipulations, including but not limited to, the ligation of two or more oligonucleotide sequences generated by restriction enzyme digestion of a polynucleotide sequence, the synthesis of oligonucleotides (e.g., the synthesis of primers or oligonucleotides) and the like.

As used herein, “recombinant PE” refers to PE in which the DNA sequence encoding the PE is modified to produce a variant (or mutant) DNA sequence which encodes the substitution, deletion or insertion of one or more amino acids in the naturally-occurring amino acid sequence. Suitable methods to produce such modification are well-known to those in the art.

The term “recombinant DNA molecule” as used herein refers to a DNA molecule that is comprised of segments of DNA joined together by means of molecular biological techniques.

As used herein, the term “enzymatic conversion” refers to the modification of a carbon substrate to an intermediate or the modification of an intermediate to an end-product by contacting the substrate or intermediate with an enzyme. In some embodiments, contact is made by directly exposing the substrate or intermediate to the appropriate enzyme. In other embodiments, contacting comprises exposing the substrate or intermediate to an organism that expresses and/or excretes the enzyme, and/or metabolizes the desired substrate and/or intermediate to the desired intermediate and/or end-product, respectively.

As used herein, “wild-type” and “native” proteins are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring (i.e., precursor) protein may be obtained in accord with the general methods known to those skilled in the art. The methods generally comprise synthesizing labeled probes having putative sequences encoding regions of the protein of interest, preparing genomic libraries from organisms expressing the protein, and screening the libraries for the gene of interest by hybridization to the probes. Positively hybridizing clones are then mapped and sequenced.

The term “sample” as used herein is used in its broadest sense. However, in preferred embodiments, the term is used in reference to a sample (e.g., an aliquot) that comprises a peptide (ie., a peptide within a pepset, that comprises a sequence of a protein of interest) that is being analyzed, identified, modified, and/or compared with other peptides. Thus, in most cases, this term is used in reference to material that includes a protein or peptide that is of interest. As used herein, “Stimulation Index” (SI) refers to a measure of the T-cell proliferative response of a peptide compared to a control. The SI is calculated by dividing the average CPM (counts per minute) obtained in testing the CD4⁺T-cell and dendritic cell culture containing a peptide by the average CPM of the control culture containing dendritic cells and CD4⁺T-cells but without the peptides. This value is calculated for each donor and for each peptide. While SI values of between about 1.5 to 4.5 may be used to indicate a positive response, the preferred SI value to indicate a positive response is between 2.5 and 3.5, inclusive, preferably between 2.7 and 3.2 inclusive, and more preferably between 2.9 and 3.1 inclusive. The most preferred embodiments described herein use a SI value of 2.95.

As used herein, the term “dataset” as used herein refers to compiled data for a set of peptides and a set of donors for each protein.

As used herein, the term “pepset” refers to the set of peptides obtained from each test protein (i.e., protein of interest). These peptides in the pepset (or “peptide sets”) are tested with cells from each donor.

As used herein, the terms “purified” and “isolated” refer to the removal of contaminants from a sample. For example, PEs are purified by removal of contaminating proteins and other compounds within a solution or preparation that are not PEs. In some embodiments, recombinant PEs are expressed in bacterial host cells and these recombinant PEs are purified by the removal of other host cell constituents; the percent of recombinant PE polypeptides is thereby increased in the sample.

As used herein, “background level” and “background response” refer to the average percent of responders to any given peptide in the dataset for any tested protein. This value is determined by averaging the percent responders for all peptides in the set, as compiled for all the tested donors. As an example, a 3% background response would indicate that on average there would be three positive (SI greater than 2.95) responses for any peptide in a dataset when tested on 100 donors.

As used herein, “antigen presenting cell” (“APC”) refers to a cell of the immune system that presents antigen on its surface, such that the antigen is recognizable by receptors on the surface of T-cells. Antigen presenting cells include, but are not limited to dendritic cells, interdigitating cells, activated B-cells and macrophages.

The term “lymphoid” when used in reference to a cell line or a cell, means that the cell line or cell is derived from the lymphoid lineage and includes cells of both the B and the T lymphocyte lineages.

As used herein, the terms “T lymphocyte” and “T-cell,” encompass any cell within the T lymphocyte lineage from T-cell precursors (including Thy1 positive cells which have not rearranged the T cell receptor genes) to mature T cells (i.e., single positive for either CD4 or CD8, surface TCR positive cells).

As used herein, the terms “B lymphocyte” and “B-cell” encompasses any cell within the B-cell lineage from B-cell precursors, such as pre-B-cells (B220⁺ cells which have begun to rearrange Ig heavy chain genes), to mature B-cells and plasma cells.

As used herein, “CD4⁺T-cell” and “CD4 T-cell” refer to helper T-cells, while “CD8⁺T-cell” and CD8 T-cell” refer to cytotoxic T-cells.

As used herein, “B-cell proliferation,” refers to the number of B-cells produced during the incubation of B-cells with the antigen presenting cells, with or without antigen.

As used herein, “baseline B-cell proliferation,” as used herein, refers to the degree of B-cell proliferation that is normally seen in an individual in response to exposure to antigen presenting cells in the absence of peptide or protein antigen. For the purposes herein, the baseline B-cell proliferation level is determined on a per sample basis for each individual as the proliferation of B-cells in the absence of antigen.

As used herein, “B-cell epitope,” refers to a feature of a peptide or protein that is recognized by a B-cell receptor in the immunogenic response to the peptide comprising that antigen (i.e., the immunogen).

As used herein, “altered B-cell epitope,” refers to an epitope amino acid sequence which differs from the precursor peptide or peptide of interest, such that the variant peptide of interest produces different (i.e., altered) immunogenic responses in a human or another animal. It is contemplated that an altered immunogenic response includes altered immunogenicity and/or allergenicity (i.e., an either increased or decreased overall immunogenic response). In some embodiments, the altered B-cell epitope comprises substitution and/or deletion of an amino acid selected from those residues within the identified epitope. In alternative embodiments, the altered B-cell epitope comprises an addition of one or more residues within the epitope.

As used herein “T-cell epitope” means a feature of a peptide or protein that is recognized by a T-cell receptor in the initiation of an immunologic response to the peptide comprising that antigen. Recognition of a T-cell epitope by a T-cell is generally believed to be via a mechanism wherein T-cells recognize peptide fragments of antigens which are bound to class I or class II Major Histocompatibility Complex (MHC) molecules expressed on antigen-presenting cells (See e.g., Moeller (ed.), Immunol. Rev., 98:187 [1987]). In some embodiments of the present invention, the epitopes or epitopic fragments identified as described herein find use in the detection of antigen presenting cells having MHC molecules capable of binding and displaying the epitopes or fragments. In some embodiments, the epitopes/epitopic fragments further comprise a detectable label (i.e., a marker) that facilitates the identification of cells that bind and/or display the epitope/epitopic fragment of interest.

As used herein, “T-cell proliferation,” refers to the number of T-cells produced during the incubation of T-cells with the antigen presenting cells, with or without antigen.

“Baseline T-cell proliferation,” as used herein, refers to the degree of T-cell proliferation that is normally seen in an individual in response to exposure to antigen presenting cells in the absence of peptide or protein antigen. For the purposes herein, the baseline T-cell proliferation level is determined on a per sample basis for each individual as the proliferation of T-cells in response to antigen presenting cells in the absence of antigen.

As used herein “altered immunogenic response,” refers to an increased or reduced immunogenic response. Proteins and peptides exhibit an “increased immunogenic response” when the T-cell and/or B-cell response they evoke is greater than that evoked by a parental (e.g., precursor) protein or peptide (e.g., the protein of interest). The net result of this higher response is an increased antibody response directed against the variant protein or peptide. Proteins and peptides exhibit a “reduced immunogenic response” when the T-cell and/or B-cell response they evoke is less than that evoked by a parental (e.g., precursor) protein or peptide. In preferred embodiments, the net result of this lower response is a reduced antibody response directed against the variant protein or peptide. In some preferred embodiments, the parental protein is a wild-type protein or peptide.

As used herein, an “in vivo reduction in immunogenicity” refers to an exhibited decrease in the immunogenic response as determined by an assay that occurs at least in part, within a living organism, (e.g., requires the use of an living animal). Exemplary “in vivo” assays include determination of altered immunogenic responses in mouse models.

As used herein, an “in vitro reduction in immunogenicity” refers an exhibited decrease in the immunogenic response as determined by an assay that occurs in an artificial environment outside of a living organism (i.e., does not require use of a living animal). Exemplary in vitro assays include testing the proliferative responses by human peripheral blood mononuclear cells to a peptide of interest.

As used herein, the term “significant epitope” refers to an epitope (i.e., a T-cell and/or B-cell epitope) wherein the response rate within the tested donor pool is equal to or greater than about three times the background response rate.

As used herein, a “weakly significant epitope” refers to an epitope (i.e., a T-cell and/or B-cell epitope), wherein the response rate within the tested donor pool is greater than the background response rate, but less than about three times the background rate.

As used herein, “protein of interest,” refers to a protein which is being analyzed, identified and/or modified. Naturally-occurring, as well as recombinant proteins find use in the present invention.

As used herein, “protein” refers to any composition comprised of amino acids and recognized as a protein by those of skill in the art. The terms “protein,” “peptide” and polypeptide are used interchangeably herein. Wherein a peptide is a portion of a protein, those skill in the art understand the use of the term in context. The term “protein” encompasses mature forms of proteins, as well as the pro- and prepro-forms of related proteins. Prepro forms of proteins comprise the mature form of the protein having a prosequence operably linked to the amino terminus of the protein, and a “pre-” or “signal” sequence operably linked to the amino terminus of the prosequence.

The variants of the present invention include the mature forms of protein variants, as well as the pro- and prepro-forms of such protein variants. The prepro-forms are the preferred construction since this facilitates the expression, secretion and maturation of the protein variants.

As used herein, “prosequence” refers to a sequence of amino acids bound to the N-terminal portion of the mature form of a protein which when removed results in the appearance of the “mature” form of the protein. Many enzymes are found in nature as translational proenzyme products and, in the absence of post-translational processing, are expressed in this fashion.

As used herein, “signal sequence” and “presequence” refer to any sequence of amino acids bound to the N-terminal portion of a protein or to the N-terminal portion of a pro-protein which may participate in the secretion of the mature or pro forms of the protein. This definition of signal sequence is a functional one and is intended to include all those amino acid sequences encoded by the N-terminal portion of the protein gene that participate in the effectuation of the secretion of protein under native conditions. The present invention utilizes such sequences to effect the secretion of the protein variants described herein.

As used herein, a “prepro” form of a protein variant consists of the mature form of the protein having a prosequence operably linked to the amino terminus of the protein and a “pre” or “signal” sequence operably linked to the amino terminus of the prosequence.

As used herein, functionally similar proteins are considered to be “related proteins.” In some embodiments, these proteins are derived from a different genus and/or species, including differences between classes of organisms (e.g., a bacterial protein and a fungal protein). In some embodiments, these proteins are derived from a different genus and/or species, including differences between classes of organisms. In additional embodiments, related proteins are provided from the same species.

As used herein, the term “derivative” refers to a protein which is derived from a precursor protein by addition of one or more amino acids to either or both the C— and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is preferably achieved by modifying a DNA sequence which encodes for the native protein, transformation of that DNA sequence into a suitable host, and expression of the modified DNA sequence to form the derivative protein.

Related (and derivative) proteins comprise “variant proteins.” In preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In one preferred embodiment, the number of different amino acids between variants is between 1 and 10. In particularly preferred embodiments, related proteins and particularly variant proteins comprise at least 50%, 60%, 65%. 70%, 75%, 80%, 85%, 90%, 95%, 97%, 98%, or 99% amino acid sequence identity. Additionally, a related protein or a variant protein as used herein, refers to a protein that differs from another related protein or a parent protein in the number of prominent regions. For example, in some embodiments, variant proteins have 1, 2, 3, 4, 5, or 10 corresponding prominent regions that differ from the parent protein.

In one embodiment, the prominent corresponding region of a variant produces only a background level of immunogenic response. Some of the residues identified for substitution, insertion or deletion are conserved residues whereas others are not. In the case of residues which are not conserved, the replacement of one or more amino acids is limited to substitutions which produce a variant which has an amino acid sequence that does not correspond to one found in nature. In the case of conserved residues, such replacements should not result in a naturally-occurring sequence.

In some embodiments, modification is preferably made to the “precursor DNA sequence” which encodes the amino acid sequence of the precursor enzyme, but can be by the manipulation of the precursor protein. In the case of residues which are not conserved, the replacement of one or more amino acids is limited to substitutions which produce a variant which has an amino acid sequence that does not correspond to one found in nature. In the case of conserved residues, such replacements should not result in a naturally-occurring sequence. Derivatives provided by the present invention further include chemical modification(s) that change the characteristics of the PE.

In some embodiments, characteristics of variant PEs are determined by methods known to those skilled in the art. Exemplary characteristics include, but are not limited to thermal stability, alkaline stability, and stability of the particular PE, various substrate, buffer solutions and/or product formulations. In combination with enzyme stability assays, variant PEs obtained through random mutagenesis can be identified which demonstrate either increased or decreased alkaline or thermal stability while maintaining enzymatic activity.

Alkaline stability can be measured either by known procedures or by the methods described herein. A substantial change in alkaline stability is evidenced by at least about a 5% or greater increase or decrease (in most embodiments, it is preferably an increase) in the half-life of the enzymatic activity of a mutant when compared to the precursor protein.

Thermal stability can be measured either by known procedures or by the methods described herein. A substantial change in thermal stability is evidenced by at least about a 5% or greater increase or decrease (in most embodiments, it is preferably an increase) in the half-life of the catalytic activity of a mutant when exposed to a relatively high temperature and neutral pH as compared to the precursor protein.

In some preferred embodiments, the PE gene is ligated into an appropriate expression plasmid. The cloned PE gene is then used to transform or transfect a host cell in order to express the PE gene. This plasmid may replicate in hosts in the sense that it contains the well-known elements necessary for plasmid replication or the plasmid may be designed to integrate into the host chromosome. The necessary elements are provided for efficient gene expression (e.g., a promoter operably linked to the gene of interest). In some embodiments, these necessary elements are supplied as the gene's own homologous promoter if it is recognized, (i.e., transcribed, by the host), a transcription terminator (a polyadenylation region for eukaryotic host cells) which is exogenous or is supplied by the endogenous terminator region of the PE gene. In some embodiments, a selection gene such as an antibiotic resistance gene that enables continuous cultural maintenance of plasmid-infected host cells by growth in antimicrobial-containing media is also included.

The following cassette mutagenesis method may be used to facilitate the construction of the PE variants of the present invention, although other methods may be used. First, the naturally-occurring gene encoding the PE is obtained and sequenced in whole or in part. Then, the sequence is scanned for a point at which it is desired to make a mutation (deletion, insertion or substitution) of one or more amino acids in the encoded PE. The sequences flanking this point are evaluated for the presence of restriction sites for replacing a short segment of the gene with an oligonucleotide pool which when expressed will encode various mutants. Such restriction sites are preferably unique sites within the protein gene so as to facilitate the replacement of the gene segment. However, any convenient restriction site which is not overly redundant in the PE gene may be used, provided the gene fragments generated by restriction digestion can be reassembled in proper sequence. If restriction sites are not present at locations within a convenient distance from the selected point (from 10 to 15 nucleotides), such sites are generated by substituting nucleotides in the gene in such a fashion that neither the reading frame nor the amino acids encoded are changed in the final construction. Mutation of the gene in order to change its sequence to conform to the desired sequence is accomplished by M13 primer extension in accord with generally known methods. The task of locating suitable flanking regions and evaluating the needed changes to arrive at two convenient restriction site sequences is made routine by the redundancy of the genetic code, a restriction enzyme map of the gene and the large number of different restriction enzymes. Note that if a convenient flanking restriction site is available, the above method need be used only in connection with the flanking region which does not contain a site.

Once the naturally-occurring DNA or synthetic DNA is cloned, the restriction sites flanking the positions to be mutated are digested with the cognate restriction enzymes and a plurality of end termini-complementary oligonucleotide cassettes are ligated into the gene. The mutagenesis is simplified by this method because all of the oligonucleotides can be synthesized so as to have the same restriction sites, and no synthetic linkers are necessary to create the restriction sites.

As used herein, “corresponding to,” refers to a residue at the enumerated position in a protein or peptide, or a residue that is analogous, homologous, or equivalent to an enumerated residue in a protein or peptide.

As used herein, “corresponding region,” generally refers to an analogous position along related proteins or a parent protein.

The terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “analogous sequence” refers to a sequence within a protein that provides similar function, tertiary structure, and/or conserved residues as the protein of interest (i.e., typically the original protein of interest). In particularly preferred embodiments, the analogous sequence involves sequence(s) at or near an epitope. For example, in epitope regions that contain an alpha helix or a beta sheet structure, the replacement amino acids in the analogous sequence preferably maintain the same specific structure. The term also refers to nucleotide sequences, as well as amino acid sequences. In some embodiments, analogous sequences are developed such that the replacement amino acids show a similar function, the tertiary structure and/or conserved residues to the amino acids in the protein of interest at or near the epitope. Thus, where the epitope region contains, for example, an alpha-helix or a beta-sheet structure, the replacement amino acids preferably maintain that specific structure.

As used herein, “homologous protein” refers to a protein (e.g. PE) that has similar action, structure, antigenic, and/or immunogenic response as a protein of interest (e.g., PE from another source, such as another strain of Pseudomonas). It is not intended that homologs be necessarily related evolutionarily. Thus, it is intended that the term encompass the same functional protein obtained from different species. In some preferred embodiments, it is desirable to identify a homolog that has a tertiary and/or primary structure similar to the protein of interest, as replacement for the epitope in the protein of interest with an analogous segment from the homolog will reduce the disruptiveness of the change. Thus, in most cases, closely homologous proteins provide the most desirable sources of epitope substitutions. Alternatively, it is advantageous to look to human analogs for a given protein. For example, in some embodiments, substituting a specific epitope in one PE with a sequence from another PE or other species' PE results in the production of PE with reduced immunogenicity. In some preferred embodiments, the PE homologs of the present invention have tertiary and/or primary structures substantially similar to wild-type PE. A significant PE epitope may be replaced with an analogous segment from a homologous enzyme. This type of replacement may reduce the disruptiveness of the change in the parent PE. In most cases, closely homologous proteins provide the most desirable source of epitope substitutions.

As used herein, “homologous genes” refers to at least a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes). These genes encode “homologous proteins.”

As used herein, “ortholog” and “orthologous genes” refer to genes in different species that have evolved from a common ancestral gene (i.e., a homologous gene) by speciation. Typically, orthologs retain the same function in during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

The degree of homology between sequences may be determined using any suitable method known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

For example, PILEUP is a useful program to determine sequence homology levels. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps. Another example of a useful algorithm is the BLAST algorithm, described by Altschul et al., (Altschul et al., J. Mol. Biol., 215:403-410, [1990]; and Karlin et al., Proc. Natl. Acad. Sci. USA 90:5873-5787 [1993]). One particularly useful BLAST program is the WU-BLAST-2 program (See, Altschul et al., Meth. Enzymol., 266:460-480 [1996]). parameters “W,” “T,” and “X” determine the sensitivity and speed of the alignment. The BLAST program uses as defaults a wordlength (W) of 11, the BLOSUM62 scoring matrix (See, Henikoff and Henikoff, Proc. Natl. Acad. Sci. USA 89:10915 [1989]) alignments (B) of 50, expectation (E) of 10, M′5, N′-4, and a comparison of both strands.

As used herein, “percent (%) nucleic acid sequence identity” is defmed as the percentage of nucleotide residues in a candidate sequence that are identical with the nucleotide residues of the sequence.

As used herein, the term “hybridization” refers to the process by which a strand of nucleic acid joins with a complementary strand through base pairing, as known in the art.

As used herein, “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes. For example, 6×SSC=very low stringency; 3×SSC =low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe.

For applications requiring high selectivity, one will typically desire to employ relatively stringent conditions to form the hybrids (e.g., relatively low salt and/or high temperature conditions are used).

The phrases “substantially similar and “substantially identical” in the context of at least two nucleic acids or polypeptides typically means that a polynucleotide or polypeptide comprises a sequence that has at least 60% identity, preferably at least 75% sequence identity, more preferably at least 80%, yet more preferably at least 90%, still more preferably 95%, most preferably 97%, sometimes as much as 98% and 99% sequence identity, compared to the reference (i.e., wild-type) sequence. Sequence identity may be determined using known programs such as BLAST, ALIGN, and CLUSTAL using standard parameters. (See e.g., Altschul, et al., J. Mol. Biol. 215:403-410 [1990]; Henikoffet al., Proc. Natl. Acad Sci. USA 89:10915 [1989]; Karin et al., Proc. Natl Acad. Sci USA 90:5873 [1993]; and Higgins et al., Gene 73:237-244 [1988]). Software for performing BLAST analyses is publicly available through the National Center for Biotechnology Information. Also, databases may be searched using FASTA (Pearson et al., Proc. Natl. Acad. Sci. USA 85:2444-2448 [1988]). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “equivalent residues” refers to proteins that share particular amino acid residues. For example, equivalent resides may be identified by determining homology at the level of tertiary structure for a protein (e.g., IFN-β) whose tertiary structure has been determined by x-ray crystallography. Equivalent residues are defined as those for which the atomic coordinates of two or more of the main chain atoms of a particular amino acid residue of the protein having putative equivalent residues and the protein of interest (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins analyzed. The preferred model is the crystallographic model giving the lowest R factor for experimental diffraction data at the highest resolution available, determined using methods known to those skilled in the art of crystallography and protein characterization/analysis.

The present invention encompasses PEs having altered immunogenicity that are equivalent to those that are derived from the particular microbial strain mentioned. Being “equivalent,” in this context, means that the PEs are encoded by a polynucleotide capable of hybridizing to the polynucleotide encoding the amino acid sequence of SEQ ID NO:1 under conditions of medium to high stringency and still retaining the altered immunogenic response to human T-cells. Thus, in some embodiments, equivalent PEs comprise at least 55%, at least 65%, at least 70%, at least 75%, at least 80%, at least 85%, at least 90%, at least 95%, at least 97% or at least 99% identity to the epitope sequences and the variant PEs having such epitopes.

As used herein, the terms “hybrid PEs” and “fusion PEs” refer to proteins that are engineered from at least two different or “parental” proteins. In preferred embodiments, these parental proteins are homologs of one another. For example, in some embodiments, a preferred hybrid PE or fusion protein contains the N-tenninus of a protein and the C-tenninus of a homolog of the protein. In some preferred embodiment, the two tenninal ends are combined to correspond to the full-length active protein. In alternative preferred embodiments, the homologs share substantial similarity but do not have identical T-cell epitopes. Therefore, in one embodiment, the present invention provides a PE of interest having one or more T-cell epitopes in the C-terminus, but in which the C-terminus is replaced with the C-terminus of a homolog having a less potent T-cell epitope, or fewer or no T-cell epitopes in the C-terminus. Thus, the skilled artisan understands that by being able to identify T-cell epitopes among homologs, a variety of variants producing different immunogenic responses can be formed. Moreover, it is understood that internal portions, and more than one homolog can be used to produce the variants of the present invention.

The term “regulatory element” as used herein refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “expression vector” refers to a DNA construct containing a DNA sequence that is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. In the present specification, “plasmid” and “vector” are sometimes used interchangeably as the plasmid is the most commonly used form of vector at present. However, the invention is intended to include such other forms of expression vectors that serve equivalent functions and which are, or become, known in the art.

As used herein, “host cells” are generally prokaryotic or eukaryotic hosts which may have been manipulated by the methods known in the art (See e.g., U.S. Pat. No. 4,760,025 (RE 34,606)) to render them incapable of secreting enzymatically active endoprotease. In some preferred embodiments, the host cell used for expressing protein is the Bacillus strain BG2036, which is deficient in enzymatically active neutral protein and alkaline protease (subtilisin). The construction of strain BG2036 is described in detail in U.S. Pat. No. 5,264,366, hereby incorporated by reference. Other host cells for expressing protein include Bacillus subtilis I168 (also described in U.S. Pat. No. 4,760,025 (RE 34,606) and U.S. Pat. No. 5,264,366, the disclosures of which are incorporated herein by reference), as well as any suitable Bacillus strain, including, but not limited to those within the species of B. licheniformis, B. lentus, and other Bacillus species, etc. However, it is not intended that the present invention be limited to Bacillus species as host cells, as other organisms are known in the art as suitable host cells (e.g., E. coli, etc.). In some preferred embodiments, the host cell has been modified such that endogenous PE is not produced by the host cell.

Host cells are transformed or transfected with vectors constructed using recombinant DNA techniques known in the art. Transformed host cells are capable of either replicating vectors encoding the protein variants or expressing the desired protein variant. In the case of vectors which encode the pre- or prepro-form of the protein variant, such variants, when expressed, are typically secreted from the host cell into the host cell medium.

The term “introduced” in the context of inserting a nucleic acid sequence into a cell, means transformation, transduction or transfection. Means of transformation include protoplast transformation, calcium chloride precipitation, electroporation, naked DNA and the like as known in the art. (See, Chang and Cohen, Mol. Gen. Genet., 168:111-115 [1979]; Smith et al., Appl. Env. Microbiol., 51:634 [1986]; and the review article by Ferrari et al., in Bacillus Harwood (ed.), Plenum Publishing Corporation, pp. 57-52 [1989]).

The term “promoter/enhancer” denotes a segment of DNA which contains sequences capable of providing both promoter and enhancer functions (for example, the long terminal repeats of retroviruses contain both promoter and enhancer functions). The enhancer/promoter may be “endogenous” or “exogenous” or “heterologous.” An endogenous enhancer/promoter is one which is naturally linked with a given gene in the genome. An exogenous (heterologous) enhancer/promoter is one which is placed in juxtaposition to a gene by means of genetic manipulation (i.e., molecular biological techniques).

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

The term “stable transfection” or “stably transfected” refers to the introduction and integration of foreign DNA into the genome of the transfected cell. The term “stable transfectant” refers to a cell which has stably integrated foreign or exogenous DNA into the genomic DNA of the transfected cell.

The terms “selectable marker” or “selectable gene product” as used herein refer to the use of a gene which encodes an enzymatic activity that confers resistance to an antibiotic or drug upon the cell in which the selectable marker is expressed.

As used herein, the terms “amplification” and “gene amplification” refer to a process by which specific DNA sequences are disproportionately replicated such that the amplified gene becomes present in a higher copy number than was initially present in the genome. In some embodiments, selection of cells by growth in the presence of a drug (e.g., an inhibitor of an inhibitable enzyme) results in the amplification of either the endogenous gene encoding the gene product required for growth in the presence of the drug or by amplification of exogenous (i.e., input) sequences encoding this gene product, or both. Gene amplification occurs naturally during development in particular genes such as the amplification of ribosomal genes in amphibian oocytes. Gene amplification may be induced by treating cultured cells with drugs. An example of drug-induced amplification is the methotrexate-induced amplification of the endogenous dhfr gene in mammalian cells (Schmike et al., Science 202:1051 [1978]). Selection of cells by growth in the presence of a drug (e.g., an inhibitor of an inhibitable enzyme) may result in the amplification of either the endogenous gene encoding the gene product required for growth in the presence of the drug or by amplification of exogenous (i.e., input) sequences encoding this gene product, or both.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “co-amplification” refers to the introduction into a single cell of an amplifiable marker in conjunction with other gene sequences (i.e., comprising one or more non-selectable genes such as those contained within an expression vector) and the application of appropriate selective pressure such that the cell amplifies both the amplifiable marker and the other, non-selectable gene sequences. The amplifiable marker may be physically linked to the other gene sequences or alternatively two separate pieces of DNA, one containing the amplifiable marker and the other containing the non-selectable marker, may be introduced into the same cell.

As used herein, the terms “amplifiable marker,” “amplifiable gene,” and “amplification vector” refer to a marker, gene or a vector encoding a gene which permits the amplification of that gene under appropriate growth conditions.

As used herein, the term “amplifiable nucleic acid” refers to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of “target” (defined below). In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Template specificity” is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (See e.g., Kacian et al., Proc. Natl. Acad. Sci. USA 69:3038 [1972]). Other nucleic acids are not replicated by this amplification enzyme. Similarly, in the case of T7 RNA polymerase, this amplification enzyme has a stringent specificity for its own promoters (See, Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (See, Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the methods of U.S. Pat. Nos. 4,683,195 4,683,202, and 4,965,188, hereby incorporated by reference, which include methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or DATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the terms “restriction endonucleases” and “restriction enzymes” refer to bacterial enzymes, each of which cut double-stranded DNA at or near a specific nucleotide sequence.

As used herein, “effective amount of PE enzyme” refers to the quantity of PE enzyme necessary to achieve the enzymatic activity required in the specific application. Such effective amounts are readily ascertained by one of ordinary skill in the art and are based on many factors, such as the particular enzyme variant used, the application involved, the specific composition of the overall composition, etc.

As used herein, “pharmaceutically-acceptable” means that drugs, medicaments and/or inert ingredients which the term describes are suitable for use in contact with the tissues of humans and other animals without undue toxicity, incompatibility, instability, irritation, allergic response, and the like, commensurate with a reasonable benefit/risk ratio.

As indicated above, the PEs of the present invention exhibit modified immunogenic responses (e.g., antigenicity and/or immunogenicity) when compared to the native PEs encoded by their precursor DNAs. In some preferred embodiments, the proteins (e.g., PEs) exhibit reduced allergenicity/immunogenicity. Those of skill in the art readily recognize that the uses of the PEs of this invention will be determined, in large part, on the immunological properties of the proteins.

In one embodiment of the present invention, the epitopes identified herein are used to elicit an immune response (e.g., where it is desired to raise antibodies against PE) including any of the epitopes described herein, as well as other epitopes known in the art. Such antibodies find use in screening for other PEs that include at least one of these regions, or regions highly homologous thereto. In addition, the PEs of the present invention find use as reagents in various assays, such as immunoassays utilizing isolated natural epitope, recombinant protein, or synthetic peptide representing specific epitopic regions to evaluate persons for sensitization to proteins including these or highly homologous regions.

In another embodiment, the epitopic fragments herein are used in the detection of antigen presenting cells having MHC molecules capable of binding and displaying such fragments. For example, the epitopic fragments can include a detectable label (e.g., radiolabel). The labeled fragments are then incubated with cells of interest, and then cells which bind (or display) the labeled fragments are detected.

In additional embodiments, the related and/or variant PEs with reduced allergenicity/immunogenicity find use in other applications, including pharmaceutical applications, drug delivery applications, cancer treatment regimens, and other health care applications.

In addition, the related and/or variant proteins with reduced allergenicity/immunogenicity find use in other applications, including pharmaceutical applications, drug delivery applications, cancer treatment regimens, and other health care applications. Indeed, it is contemplated that the PEs of the present invention will find widespread use in numerous compositions and applications.

### DETAILED DESCRIPTION OF THE INVENTION

The present invention provides PE CD4+T-cell epitopes, as well as novel variants that exhibit reduced immunogenic responses, as compared to the parental PE. The present invention further provides DNA molecules that encode novel PE variants, and host cells comprising DNA encoding novel PE variants, as well as methods for making PEs less immunogenic. In addition, the present invention provides various compositions that comprise these PE variants that are less immunogenic than the wild-type PEs. In some specific embodiments, the present invention provides PE variants with reduced immunogenicity that are identified and/or characterized using the methods of the present invention.

Following the general assessment of the PE38 T-cell epitopes, four T-cell epitopes in the PE38 were further analyzed as described using the methods of the present invention. The following epitopes (one major and two minor epitopes) determined to be of interest are listed in Table 1, below. Peptide 75 has the sequence ARGRIRNGALLRVY (SEQ ID NO:5), while peptide 76 has the sequence RIRNGALLRVYVPRS (SEQ ID NO:6). Peptide “75-76” is a combination of peptides 75 and 76, as indicated in Table 1.

TABLE 1

Peptides of Interest in PE

| Peptide | | |
|---|---|---|
| number | Sequence | SEQ ID NO: |
| 75-76 | ARGRIRNGALLRVYVPRS | SEQ ID NO: 2 |
| (15) | RLVALYLAARLSWNQ | SEQ ID NO: 3 |
| (65) | SQDLDAIWRGFYIAG | SEQ ID NO: 4 |

In some embodiments, the present invention further includes identifying residues that decrease the immunogenicity of PE. In some embodiments, at least one amino acid substitution is made in at least one T-cell epitope in the wild-type or parental PE sequence. In some preferred embodiments, multiple amino acids are changed in the parental PE sequence to produce a PE variant having reduced allergenicity/immunogenicity. In alternative preferred embodiments, amino acid deletions, insertions and/or substitutions are made in the parental PE sequence to produce a variant PE with reduced allergenicity/immunogenicity. In some embodiments, this PE is wild-type, while in other embodiments, it is a mutated variant, conjugated variant, or a hybrid variant having amino acid substitutions in the epitope of interest.

In addition to PEs having reduced allergenicity/immunogenicity, the present invention encompasses variant PEs having increased immunogenicity, as well as variant PEs having the same degree of allergenicity/immunogenicity as the parental PE, but with other altered characteristics. In alternative preferred embodiments, amino acid deletions, insertions and/or substitutions are made in the parental PE sequence to produce a variant PE with increased or decreased allergenicity/immunogenicity. In additional embodiments, the variant PE comprises alterations in the non-T-cell epitope regions of the amino acid sequences. Thus, the present invention encompasses PEs with the same reactivity toward T-cells, but with other altered characteristics. In preferred embodiments, these altered characteristics provide beneficial features to the variant PE. In some embodiments, the parental PE is wild-type, while in other embodiments, it is a mutated variant, conjugated variant, or a hybrid variant having amino acid substitutions in the epitope of interest.

In one preferred embodiment of the present invention, a peptide having an altered immunogenic response, (e.g., increased or decreased immunogenic response) is derived from a PE of interest. In some embodiments, the epitope is identified by an assay which identifies epitopes and non-epitopes as follows: differentiated dendritic cells are combined with naive human CD4+ and/or CD8+ T-cells and with a peptide of interest. More specifically, in some embodiments, a reduced immunogenic response peptide of interest is provided wherein a T-cell epitope is recognized comprising the steps of: (a) obtaining from a single blood source a solution of dendritic cells and a solution of naive CD4+ and/or CD8+T-cells; (b) differentiating the dendritic cells; (c) combining the solution of differentiated dendritic cells and the naive CD4+ and/or CD8+T-cells with a peptide of interest; and (d) measuring the proliferation of T-cells in step (c).

In some preferred embodiments of the present invention, a series of peptide oligomers which correspond to all or part of the PE of interest are prepared. In some particularly preferred embodiments, a peptide library is produced covering the relevant portion or all of the protein. As described in the Examples below, a set of 15-mer peptides offset by three amino acids was used to identify the epitopes of interest. By analyzing each of the peptides individually in the assay provided herein, the methods of the present invention facilitate the precise identification of epitopes recognized by T-cells. In the example above, the greater reaction of one specific peptide than its neighbors facilitates identification of the epitope anchor region to within three amino acids. In some embodiments, after the locations of these epitopes are determined, one or more of the amino acids within at least one epitope are modified, until the peptide produces a different T-cell response from that of the original protein.

The present invention extends to all proteins against which it is desired to modulate the immunogenic response. Those of skill in the art readily recognize the proteins and peptides of this invention are not necessarily native proteins and peptides. Indeed, in one embodiment of the present invention, shuffled genes having an altered immunologic response are contemplated (See e.g., Stemmer, Proc. Natl. Acad. Sci. USA 91:10747 [1994]; Patten et al., Curr. Op. Biotechnol.8:724 [1997]; Kuchner and Arnold, Trends Biotechnol., 15:523 [1997]; Moore et al., J. Mol. Biol., 272:336 [1997]; Zhao et al., Nature Biotechnol., 16: 258 [1998]; Giver et al., Proc. Nat'l Acad. Sci. USA 95:12809 [1998]; Harayama, Trends Biotechnol., 16:76 [1998]; Lin et al., Biotechnol., Prog., 15:467 [1999]; and Sun, Comput. Biol., 6:77 [1999], for descriptions of gene shuffling and expression of such genes). In some embodiments, PEs are altered such that the immune response generated by an animal against the PE is altered.

Preferably, PEs according to the present invention are isolated or purified. By “purification” (or “isolation”) it is meant that the PE is altered from its natural state by virtue of separating the PE from some or all of the naturally occurring constituents with which it is associated in nature. It is intended that this purification be accomplished by any suitable means known in the art, including but not limited to art-recognized separation techniques such as ion exchange chromatography, affinity chromatography, hydrophobic separation, dialysis, ammonium sulfate precipitation or other protein salt precipitation, centrifugation, size exclusion chromatography, filtration, microfiltration, gel electrophoresis or separation on a gradient to remove whole cells, cell debris, impurities, extraneous proteins, and/or enzymes undesired in the final composition. In some embodiments, constituents are added to the PE-containing composition to provide additional benefits, for example, activating agents, anti-inhibition agents, desirable ions, compounds to control pH, and/or other enzymes. In further embodiments, recombinant PE is purified.

In addition to the above proteins and peptides, the present invention encompasses wild-type PEs, as well as variant PEs that exhibit an altered immunogenic response (e.g., an increased or reduced immunogenic response). In some embodiments, variant PEs exhibit increased immunogenic responses when the T-cell response they evoke is greater than that evoked by a parental (i.e., precursor) PE. The net result of this higher response is an increase in the antibodies directed against the variant PE. These antibodies find use in various settings, including, but not limited to methods to detect PE, PE variants, etc. In alternative embodiments, variant PEs exhibit decreased immunogenic response when the T-cell response they evoke is less than that evoked by a parental (i.e., precursor) PE. The net result of this higher response is a decrease in the antibodies directed against the variant PE. These variant PEs find use in various settings, including but not limited to administration to human and/or other animals with or without additional proteins.

Exemplary assays useful in ascertaining the reduced immunogenic response of the variant proteins include, but are not limited to in vivo assays (e.g., HLA-DR3/DQ2 mouse T cell responses, and in vitro assays (e.g., human peripheral blood mononuclear cells (PBMC)) to a sample of PE and/or PE variants. In vivo assays useful in ascertaining the reduced immunogenic response include, but are not limited to the use of transgenic mice, for example, rats (Taurog et al., Immunol. Rev., 169:209-223 [1999]), rabbits, or pigs. A preferred transgenic mouse model for testing modified proteins of interest and variants in vivo, determining a reduced immunogenic response, is the HLA-DR3/DQ2 mouse model known in the art.

In addition to altering (e.g., increasing or decreasing) the immunogenic response of an animal, such as a human, to naturally occurring amino acid sequences, the present invention provides means for reducing the immunogenic response of mutated proteins (e.g., a PE that has been altered to change the functional activity of the protein). In some embodiments, the mutation is made in order to provide some beneficial characteristic, including but not limited to increased activity, increased thermal stability, increased alkaline stability, and/or oxidative stability, etc. In some embodiments, the mutation results in the incorporation of new T-cell epitope in the mutated PE. As discussed in greater detail herein, the present invention provides PE T-cell epitopes and variant PEs with amino acid alterations that will alter the immunogenic response of the mutated protein.

Although this invention encompasses the above proteins and many others, for the sake of simplicity, the following will describe particularly preferred embodiments of the invention, which involve the modification of PE.

The amino acid position numbers used herein refer to those assigned to the PE38 sequence set forth in SEQ ID NO: 1. The invention, however, is not limited to the mutation of this particular PE but extends to precursor PEs containing amino acid residues at positions that are “equivalent” to the particular identified residues in the PE of SEQ ID NO: 1.

A residue (amino acid) of a precursor PE is equivalent to a residue of a parental PE if it is either homologous (i.e., corresponding in position in either primary or tertiary structure) or analogous to a specific residue or portion of that residue in the parental PE (i.e., having the same or similar functional capacity to combine, react, or interact chemically). “Corresponding,” as used herein generally refers to an analogous position along the peptide.

In some preferred embodiments, in order to establish homology to primary structure, the amino acid sequence of a precursor PE is directly compared to the parental PE primary sequence and particularly to a set of residues known to be invariant in PEs for which the sequence is known. After aligning the conserved residues, allowing for necessary insertions and deletions in order to maintain alignment (i.e., avoiding the elimination of conserved residues through arbitrary deletion and insertion), the residues equivalent to particular amino acids in the primary sequence of the parental PE are defined. Alignment of conserved residues preferably should conserve 100% of such residues. However, alignment of greater than 75% or as little as 50% of conserved residues is also adequate to define equivalent residues. These conserved residues, thus, may be used to define the corresponding equivalent amino acid residues of parental PE in other PEs which are highly homologous to the preferred parental PE.

In some embodiments, the present invention provides variant PEs having altered immunogenic response potential as compared to the precursor PE(s). While the instant invention is useful to lower the immunogenic response, the mutations specified herein find use in combination with mutations known in the art to result altered thermal stability and/or altered substrate specificity, modified activity, improved specific activity or altered alkaline stability as compared to the precursor.

In additional embodiments, two homologous proteins are fused so as to eliminate at least one T-cell epitope. As is described below, a region of a protein in which a T-cell epitope resides are replaced with the same region in a homologous protein that does not have the T-cell epitope. For example, a fusion protein is created with the parental PE and a homolog, so that the resulting protein does not have the T-cell epitope present in the parental PEB Any amino acid length can be used for fusion into the parental protein, from only the epitope to the majority of the protein, as long as the desired activity is maintained. However, it is not necessary that the original level of activity be maintained. Because of the lowered allergenicity/immunogenicity of the protein, in some embodiments, more of the hybrid protein is used than the parental protein, in order to achieve the same activity levels.

In some embodiments, the variant PE activity is determined and compared with the PE of interest by examining the interaction of the PE with various commercial substrates. As desired, PE activity can be determined by any suitable method known to those in the art. In additional embodiments, the other characteristics of the variant PEs are determined using suitable methods known to those in the art. Exemplary characteristics include, but are not limited to thermal stability, alkaline stability, and stability of the particular PE in various substrate or buffer solutions or product formulations. When combined with the enzyme stability assay procedure disclosed herein, mutants obtained by random mutagenesis are identified which demonstrated either increased or decreased alkaline or thermal stability while maintaining enzymatic activity.

In some embodiments, the present invention provides PE compositions with reduced immunogenicity, further comprising recognition sites for antibodies and/or cell receptors. In some preferred embodiments, the reduced immunogenicity PE is conjugated to another molecule, e.g. at least a portion of an antibody, e.g. an anti-CD22 antibody, an anti-mesothelin antibody, an anti-CD25 antibody, or an anti-Lewis Y antibody. This embodiment finds particular use in the targeted recognition of cells. In some embodiments, the cells recognized by the antibody are abnormal cells, including but not limited to malignant cells. In some embodiments, the PE is modified so as to contain at least one cancer-targeting sequence. In this embodiment, the patient (e.g., cancer patient) is administered a PE sequence that has been modified so as to directly recognize abnormal (e.g., cancer) cells. Thus, the present invention provides means to directly target cells for destruction and removal.

Suitable anti-CD22 antibody molecules useful in conjugates with PE of the present invention include RFB4 and variants thereof as described for example in WO98/41641, WO03/027135, Salvatore et al. Clin Cancer Res 2002;8:995-1002, Bang et al. Clinical Cancer Research 1545 Vol. 11, 1545-1550, Feb. 15, 2005 and Kreitman et al. J Clin Oncol 2005: 23: 6719-6729.

Suitable anti-mesothelin antibody molecules useful in conjugates with PE of the present invention include SS and SS1 as described in WO99/28471, WO0/073346 and Kreitman et al 2002: Proc. Am. Soc. Clin. Oncol. 21, 22b.

Suitable anti-CD25 antibody molecules useful in conjugates with PE of the present invention include anti-tac as described in Chaudliary et al 1989: Nature 339: 394-397.

Suitable anti-Lewis Y antibodies useful in conjugates with PE of the present invention include MAb B3 and BR96 as described in Pai et al 1996: Nature Med 2: 350-353 and Posey et al 2002: Clin. Cancer Res. 8: 3092-3099.

Other molecules suitable for cancer targeting include growth factors or cytokines e.g. IL-2, IL-4, TGFα, GMCSF, or IL-13.

Conjugates may be prepared by methods known to the person skilled in the art. Coupling of a molecule such as an antibody molecule with a PE of the invention may be directly or by the intermediary of a spacer group or of a linking group, such as a polyaldehyde, like glutaraldehyde, ethylenediaminetetraacetic acid (EDTA), diethylene-triaminepentaacetic acid (DPTA). Conjugation may be by chemical coupling, or via a covalent bond, e.g. a disulphide bond or a peptide bond, whether directly or via a peptide linker. A conjugate according to the invention may be produced as a fusion protein by expression from encoding nucleic acid. Where one component of the conjugate is a multi-chain molecule, e.g. in antibody formats such as Fab and IgG, conjugation (whether via a peptide bond or otherwise) may be to one, more than one or all of the polypeptide chains of the multi-chain molecule.

See e.g. Pastan et al 2006: Nature Reviews Cancer 6: 559-565 for a review of conjugate therapy in cancer, including discussion of different antibodies and other molecules used for targeting, different conjugates and different toxins.

### Experimental

The following examples serve to illustrate certain preferred embodiments and aspects of the present invention and are not to be construed as limiting the scope thereof.

In the experimental disclosure which follows, the following abbreviations apply: sd and SD (standard deviation); M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); LF (lethal factor); ° C. (degrees Centigrade); cDNA (copy or complimentary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); g (gravity); OD (optical density); CPM and cpm (counts per minute); rpm (revolutions per minute); Dulbecco's phosphate buffered solution (DPBS); HEPES (N-[2-Hydroxyethyl]piperazine-N-[2-ethanesulfonic acid]); HBS (HEPES buffered saline); SDS (sodium dodecylsulfate); Tris-HCl (tris[Hydroxymethyl]aminomethane-hydrochloride); 2-ME (2-mercaptoethanol); EGTA (ethylene glycol-bis(β-aminoethyl ether) N,N,N′,N′-tetraacetic acid); EDTA (ethylenediaminetetracetic acid); SI (stimulation index); PBMC (peripheral blood mononuclear cell); Endogen (Endogen, Woburn, Mass.); CytoVax (CytoVax, Edmonton, Canada); Wyeth-Ayerst (Wyeth-Ayerst, Philadelphia, Pa.); NEN (NEN Life Science Products, Boston, Mass.); Wallace Oy (Wallace Oy, Turku, Finland); Phalma AS (Pharma AS, Oslo, Norway); Dynal (Dynal, Oslo, Norway); Abbott (Abbott Laboratories, Abbott Park, Ill.); Bio-Synthesis (Bio-Synthesis, Lewisville, Tex.); ATCC (American Type Culture Collection, Rockville, Md.); Gibco/BRL (Gibco/BRL, Grand Island, N.Y.); Insightful (Insightful, Seattle, Wash.); Sigma (Sigma Chemical Co., St. Louis, Mo.); Pharmacia (Pharmacia Biotech, Piscataway, N.J.); and Stratagene (Stratagene, La Jolla, Calif.).

### Peptides

All peptides were obtained from a commercial source (Mimotopes, San Diego, Calif.). For the I-MUNE® assay system described herein, 15-mer peptides offset by 3 amino acids that described the is entire sequence of the proteins of interest were synthesized in a multipin format (See, Maeji et al., J. Immunol. Meth., 134:23-33 [1990]). Peptides were resuspended in DMSO at approximately 1 to 2 mg/ml, and stored at −70° C. prior to use. Each peptide was tested at least in duplicate. The results for each peptide were averaged. In some cases, the stimulation index (SI) was calculated for each peptide.

### Human Donor Blood Samples

Volunteer donor human blood buffy coat samples were obtained from two commercial sources (Stanford Blood Center, Palo Alto, Calif., and the Sacramento Medical Foundation, Sacramento, Calif.). Buffy coat samples were further purified by density separation. Each sample was HLA typed for HLA-DR and HLA-DQ using a commercial PCR-based kit (Bio-Synthesis). The HLA DR and DQ expression in the donor pool was determined to not be significantly different from a North American reference standard (Mori et al., Transplant., 64:1017-1027 [1997]). However, the donor pool did show evidence of slight enrichments for ethnicities common to the San Francisco Bay Area.

### Data Analysis

For each individual buffy coat sample, the average CPM values for all of the peptides were analyzed. The average CPM values for each peptide were divided by the average CPM value for the control (DMSO only) wells to determine the “stimulation index” (SI). Donors were tested with each peptide set until an average of at least two responses per peptide were compiled. The data for each protein was graphed showing the percent responders to each peptide within the set. A positive response was collated if the SI value was equal to or greater than 2.95. This value was chosen as it approximates a difference of three standard deviations in a normal population distribution. For each protein assessed, positive responses to individual peptides by individual donors were compiled. To determine the background response for a given protein, the percent responders for each peptide in the set were averaged and a standard deviation was calculated. Statistical significance was calculated using Poisson statistics for the number of responders to each peptide within the dataset. Different statistical methods were used as described herein. The response to a peptide was considered significant if either the number of donors responding to the peptide was different from the Poisson distribution defined by the dataset with a p<0.05 and/or if the percent response was at least 3-fold greater than the background.

### Statistical Methods

Statistical significance of peptide responses were calculated based on Poisson statistics. The average frequency of responders was used to calculate a Poisson distribution based on the total number of responses and the number of peptides in the set. A response was considered significant if p<0.05. In addition, two-tailed Student's t-tests with unequal variance, were performed. For epitope determination using data with low background response rates, a conservative Poisson based formula was applied:

$$= 1 - e ( - n ( 1 - \sum \frac{{λ}^{x} {e}^{- λ}}{x !} ) )$$

where n=the number of peptides in the set, x=the frequency of responses at the peptide of interest, and λ=the median frequency of responses within the dataset. For epitope determinations based on data with a high background response rate, the less stringent Poisson based determination

$$1 - ( {\sum }_{i = 0}^{x} \frac{{λ}^{x} {e}^{- λ}}{x !} )$$

was used, where λ=the median frequency of responses in the dataset, and x=the frequency of responses at the peptide of interest.

In additional embodiments, the structure determination is calculated based on the following formula:

$$\sum \vert f ( i ) - \frac{1}{p} \vert$$

wherein Σ (upper case sigma) is the sum of the absolute value of the frequency of responses to each peptide minus the frequency of that peptide in the set; f(i) is defined as the frequency of responses for an individual peptide; and p is the number of peptides in the peptide set.

This equation returns a value between 0 and 2, which is equal to the “Structure Value.” A value of 0 indicates that the results are completely without structure, and a value of 2.0 indicates all structure is highly structured around a single area. The closer the value is to 2.0, the more immunogenic the protein. Thus, a low value indicates a less immunogenic protein.

HLA Types within the Donor Pool

HLA-DR and DQ types were analyzed for associations with responses to defined epitope peptides. A Chi-squared analysis, with one degree of freedom was used to determine significance. Where an allele was present in both the responder and non-responder pools, a relative risk was calculated.

The HLA-DRB1 allelic expression was determined for approximately 84 random individuals. HLA typing was performed using low-stringency PCR determinations. PCR reactions were performed as directed by the manufacturer (Bio-Synthesis). The data compiled for the Stanford and Sacramento samples were compared the “Caucasian” HLA-DRB1 frequencies as published (See, Marsh et al., HLA Facts Book, The, Academic Press, San Diego, Calif. [2000], page 398, FIG. 1). The donor population in these communities is enriched for HLA-DR4 and HLA-DR15. However, the frequencies of these alleles in these populations are well within the reported range for these two alleles (5.2 to 24.8% for HLA-DR4 and 5.7 to 25.6% for HLA-DR15). Similarly, for HLA-DR3, -DR7 and DR11, the frequencies are lower than the average Caucasian frequency, but within the reported ranges for those alleles. Also of note, HLA DR15 is found at a higher frequency in ethnic populations that are heavily represented in the San Francisco Bay Area.

### EXAMPLE 1

### Preparation of Cells Used in the I-MUNE® Assay System for the Identification of Peptide T-Cell Epitopes in PE Using Human T-Cells

Fresh human peripheral blood cells were collected from 69 humans of unknown exposure status to PE. These cells were tested to determine antigenic epitopes in PE, as described in Example 3.

Peripheral mononuclear blood cells (stored at room temperature, no older than 24 hours) were prepared for use as follows. For each sample, approximately 30 mls of a solution of buffy coat preparation from one unit of whole blood was brought to 50 ml with Dulbecco's phosphate buffered solution (DPBS) and split into two tubes. The samples were underlaid with 12.5 ml of room temperature Lymphoprep density separation media (Nycomed; Pharma AS; Density 1.077 g/ml). The tubes were centrifuged for thirty minutes at 600×g. The interface of the two phases was collected, pooled and washed in DPBS. The cell density of the resultant solution was measured by hemocytometer, as known in the art. Viability was measured by trypan blue exclusion, as known in the art.

From the resulting solution, a differentiated dendritic cell culture was prepared from the peripheral blood mononuclear cell sample having a density of 10⁸ cells per 75 ml culture flask in a solution as described below:
(1) 50 ml of serum free AIM V media (Gibco) was supplemented with a 1:1000 dilution beta-mercaptoethanol (Gibco). The flasks were laid flat for two hours at 37° C. in 5% CO₂ to allow adherence of monocytes to the flask wall.
(2) Differentiation of the monocyte cells to dendritic cells was performed as follows: nonadherent cells were removed and the resultant adherent cells (monocytes) combined with 30 ml of AIM V, 800 units/ml of GM-CSF (Endogen) and 500 units/ml of IL-4 (Endogen); the resulting mixture was cultured for 5 days at 37° C. in 5% CO₂. After the five is days of incubation, the cytokine TNFα (Endogen) was added to 0.2 units/ml, and the cytokine IL-1α (Endogen) was added to a final concentration of 50 units/ml and the mixture incubated at 37° C. in 5% CO₂ for two more days.
(3) On the seventh day, mitomycin C was added to a concentration of 50 micrograms/ml in 100 mM EDTA-containing PBS to stop growth of the now differentiated dendritic cell culture. The solution was incubated for 60 minutes at 37° C. in 5% CO₂. Dendritic cells were dislodged from the plastic surface by gently rapping the flask. Dendritic cells were then centrifuged at 600×g for 5 minutes, washed in DPBS and counted as described above.
(4) The prepared dendritic cells were placed into a 96 well round bottom plate at a concentration of 2×10⁴ cells/well in 100 microliter total volume of AIM V media, per well.

CD4+T cells were prepared from frozen aliquots of the peripheral blood cell samples used to prepare the dendritic cells, using reagents provided by the Dynal CD4+T-cell enrichment kit (Dynal). The resultant CD4+ cell solution was centrifuged, resuspended in AIM V media and the cell density was determined using methods known in the art. The CD4+T-cell suspension was then resuspended to a count of 2×10⁶/ml in AIM V media to facilitate efficient manipulation of 96-well plates.

### EXAMPLE 2

### Identification of T-Cell Epitopes in PE

Peptides for use in the I-MUNE® assay described in Example 3 were prepared based on the sequence of PE-38 obtained from PE GenBank Accession No.AAB59097, but starting at amino acid 251 of the deposited sequence and with the deletion of amino acids 365-380 in the deposited sequence. Thus, the sequence used in these experiments had the following sequence:

(SEQ ID NO: 1)

P E G G S L A A L T A H Q A C H L P L E T F T R H
R Q P R G W E Q L E Q C G Y P V Q R L V A L Y L A
A R L S W N Q V D Q V I R N A L A S P G S G G D L
G E A I R E Q P E Q A R L A L T L A A A E S E R F
V R Q G T Q N D E A G A A N G P A D S G D A L L E
R N Y P T G A E F L G D G G D V S F S T R G T Q N
W T V E R L L Q A H R Q L E E R G Y V F V G Y H G
T F L E A A Q S I V F G G V R A R S Q D L D A I W
R G F Y I A G D P A L A Y G Y A Q D Q E P D A R G
R I R N G A L L R V Y V P R S S L P G F Y R T S L
T L A A P E A A G E V E R L I G H P L P L R L D A
I T G P E E E G G R L E T I L G W P L A E R T V V
I P S A I P T D P R N V G G D L D P S S I P D K E
Q A I S A L P D Y A S Q P G K P P R E D L K

Based upon the amino acid sequence of PE38 described above, a set of 15mers off-set by three amino acids comprising the entire sequence of PE were synthetically prepared by Mimotopes. Additional peptides are included in this table. The sequences of these peptides are provided below:

TABLE 2

Peptides Tested in I-MUNE ® Assay

| | |
|---|---|
| SEQUENCE | SEQUENCE ID NO. |

| | |
|---|---|
| ASSSVSYMHWLQQKP | SEQ ID NO: 7 |
| SVSYMHWLQQKPGTS | SEQ ID NO: 8 |
| YMHWLQQKPGTSPKL | SEQ ID NO: 9 |
| WLQQKPGTSPKLVIY | SEQ ID NO: 10 |
| QKPGTSPKLVIYLTS | SEQ ID NO: 11 |
| GTSPKLVIYLTSNLA | SEQ ID NO: 12 |
| PKLVIYLTSNLASGV | SEQ ID NO: 13 |
| VIYLTSNLASGVPAR | SEQ ID NO: 14 |
| LTSNLASGVPARFSG | SEQ ID NO: 15 |
| ASSSVSYMHWFQQKP | SEQ ID NO: 16 |
| SVSYMHWFQQKPGTS | SEQ ID NO: 17 |
| YMHWFQQKPGTSPKL | SEQ ID NO: 18 |
| WFQQKPGTSPKLVIY | SEQ ID NO: 19 |
| QKPGTSPKLVIYSTS | SEQ ID NO: 20 |
| GTSPKLVIYSTSNLA | SEQ ID NO: 21 |
| PKLVIYSTSNLASGV | SEQ ID NO: 22 |
| VIYSTSNLASGVPAR | SEQ ID NO: 23 |
| STSNLASGVPARFSG | SEQ ID NO: 24 |
| MEVQLVESGGGLVKP | SEQ ID NO: 25 |
| QLVESGGGLVKPGGS | SEQ ID NO: 26 |
| ESGGGLVKPGGSLKL | SEQ ID NO: 27 |
| GGLVKPGGSLKLSCA | SEQ ID NO: 28 |
| VKPGGSLKLSCAASG | SEQ ID NO: 29 |
| GGSLKLSCAASGFAF | SEQ ID NO: 30 |
| LKLSCAASGFAFSIY | SEQ ID NO: 31 |
| SCAASGFAFSIYDMS | SEQ ID NO: 32 |
| ASGFAFSIYDMSWVR | SEQ ID NO: 33 |
| FAFSIYDMSWVRQTP | SEQ ID NO: 34 |
| SIYDMSWVRQTPEKC | SEQ ID NO: 35 |
| DMSWVRQTPEKCLEW | SEQ ID NO: 36 |
| WVRQTPEKCLEWVAY | SEQ ID NO: 37 |
| QTPEKCLEWVAYISS | SEQ ID NO: 38 |
| EKCLEWVAYISSGGG | SEQ ID NO: 39 |
| LEWVAYISSGGGTTY | SEQ ID NO: 40 |
| VAYISSGGGTTYYPD | SEQ ID NO: 41 |
| ISSGGGTTYYPDTVK | SEQ ID NO: 42 |
| GGGTTYYPDTVKGRF | SEQ ID NO: 43 |
| TTYYPDTVKGRFTIS | SEQ ID NO: 44 |
| YPDTVKGRFTISRDN | SEQ ID NO: 45 |
| TVKGRFTISRDNAKN | SEQ ID NO: 46 |
| GRFTISRDNAKNTLY | SEQ ID NO: 47 |
| TISRDNAKNTLYLQM | SEQ ID NO: 48 |
| RDNAKNTLYLQMSSL | SEQ ID NO: 49 |
| AKNTLYLQMSSLKSE | SEQ ID NO: 50 |
| TLYLQMSSLKSEDTA | SEQ ID NO: 51 |
| LQMSSLKSEDTAMYY | SEQ ID NO: 52 |
| SSLKSEDTAMYYCAR | SEQ ID NO: 53 |
| KSEDTAMYYCARHSG | SEQ ID NO: 54 |
| DTAMYYCARHSGYGT | SEQ ID NO: 55 |
| MYYCARHSGYGTHWG | SEQ ID NO: 56 |
| CARHSGYGTHWGVLF | SEQ ID NO: 57 |
| HSGYGTHWGVLFAYW | SEQ ID NO: 58 |
| YGTHWGVLFAYWGQG | SEQ ID NO: 59 |
| HWGVLFAYWGQGTLV | SEQ ID NO: 60 |
| VLFAYWGQGTLVTVS | SEQ ID NO: 61 |
| AYWGQGTLVTVSAKA | SEQ ID NO: 62 |
| GQGTLVTVSAKASGG | SEQ ID NO: 63 |
| TLVTVSAKASGGPEQ | SEQ ID NO: 64 |
| TVSAKASGGPEGGSL | SEQ ID NO: 65 |
| AKASGGPEGGSLAAL | SEQ ID NO: 66 |
| SGGPEGGSLAALTAH | SEQ ID NO: 67 |
| PEGGSLAALTAHQAC | SEQ ID NO: 68 |
| GSLAALTAHQACHLP | SEQ ID NO: 69 |
| AALTAHQACHLPLET | SEQ ID NO: 70 |
| TAHQACHLPLETFTR | SEQ ID NO: 71 |
| QACHLPLETFTRHRQ | SEQ ID NO: 72 |
| HLPLETFTRHRQPRG | SEQ ID NO: 73 |
| LETFTRHRQPRGWEQ | SEQ ID NO: 74 |
| FTRHRQPRGWEQLEQ | SEQ ID NO: 75 |
| HRQPRGWEQLEQCGY | SEQ ID NO: 76 |
| PRGWEQLEQCGYPVQ | SEQ ID NO: 77 |
| WEQLEQCGYPVQRLV | SEQ ID NO: 78 |
| LEQCGYPVQRLVALY | SEQ ID NO: 79 |
| CGYPVQRLVALYLAA | SEQ ID NO: 80 |
| PVQRLVALYLAARLS | SEQ ID NO: 81 |
| RLVALYLAARLSWNQ | SEQ ID NO: 3 |
| ALYLAARLSWNQVDQ | SEQ ID NO: 83 |
| LAARLSWNQVDQVIR | SEQ ID NO: 84 |
| RLSWNQVDQVIRNAL | SEQ ID NO: 85 |
| WNQVDQVIRNALASP | SEQ ID NO: 86 |
| VDQVIRNALASPGSG | SEQ ID NO: 87 |
| VIRNALASPGSGGDL | SEQ ID NO: 88 |
| NALASPGSGGDLGEA | SEQ ID NO: 89 |
| ASPGSGGDLGEAIRE | SEQ ID NO: 90 |
| GSGGDLGEAIREQPE | SEQ ID NO: 91 |
| GDLGEAIREQPEQAR | SEQ ID NO: 92 |
| GEAIREQPEQARLAL | SEQ ID NO: 93 |
| IREQPEQARLALTLA | SEQ ID NO: 94 |
| QPEQARLALTLAAAE | SEQ ID NO: 95 |
| QARLALTLAAAESER | SEQ ID NO: 96 |
| LALTLAAAESERFVR | SEQ ID NO: 97 |
| TLAAAESERFVRQGT | SEQ ID NO: 98 |
| AAESERFVRQGTGND | SEQ ID NO: 99 |
| SERFVRQGTGNDEAG | SEQ ID NO: 100 |
| FVRQGTGNDEAGAAN | SEQ ID NO: 101 |
| QGTGNDEAGAANGPA | SEQ ID NO: 102 |
| GNDEAGAANGPADSG | SEQ ID NO: 103 |
| EAGAANGPADSGDAL | SEQ ID NO: 104 |
| AANGPADSGDALLER | SEQ ID NO: 105 |
| GPADSGDALLERNYP | SEQ ID NO: 106 |
| DSGDALLERNYPTGA | SEQ ID NO: 107 |
| DALLERNYPTGAEFL | SEQ ID NO: 108 |
| LERNYPTGAEFLGDG | SEQ ID NO: 109 |
| NYPTGAEFLGDGGDV | SEQ ID NO: 110 |
| TGAEFLGDGGDVSFS | SEQ ID NO: 111 |
| EFLGDGGDVSFSTRG | SEQ ID NO: 112 |
| GDGGDVSFSTRGTQN | SEQ ID NO: 113 |
| GDVSFSTRGTQNWTV | SEQ ID NO: 114 |
| SFSTRGTQNWTVERL | SEQ ID NO: 115 |
| TRGTQNWTVERLLQA | SEQ ID NO: 116 |
| TQNWTVERLLQAHRQ | SEQ ID NO: 117 |
| WTVERLLQAHRQLEE | SEQ ID NO: 118 |
| ERLLQAHRQLEERGY | SEQ ID NO: 119 |
| LQAHRQLEERGYVFV | SEQ ID NO: 120 |
| HRQLEERGYVFVGYH | SEQ ID NO: 121 |
| LEERGYVFVQYHGTF | SEQ ID NO: 122 |
| RGYVFVGYHGTFLEA | SEQ ID NO: 123 |
| VFVGYHGTFLEAAQS | SEQ ID NO: 124 |
| GYHGTFLEAAQSIVF | SEQ ID NO: 125 |
| GTFLEAAQSIVFGGV | SEQ ID NO: 126 |
| LEAAQSIVFGGVRAR | SEQ ID NO: 127 |
| AQSIVFGGVRARSQD | SEQ ID NO: 128 |
| IVFGGVRARSQDLDA | SEQ ID NO: 129 |
| GGVRARSQDLDAIWR | SEQ ID NO: 130 |
| RARSQDLDAIWRGFY | SEQ ID NO: 131 |
| SQDLDAIWRGFYIAG | SEQ ID NO: 4 |
| LDAIWRGFYIAGDPA | SEQ ID NO: 132 |
| IWRGFYIAGDPALAY | SEQ ID NO: 133 |
| GFYIAGDPALAYGYA | SEQ ID NO: 134 |
| IAGDPALAYGYAQDQ | SEQ ID NO: 135 |
| DPALAYGYAQDQEPD | SEQ ID NO: 136 |
| LAYGYAQDQEPDARG | SEQ ID NO: 137 |
| GYAQDQEPDARGRIR | SEQ ID NO: 138 |
| QDQEPDARGRIRNGA | SEQ ID NO: 139 |
| EPDARGRIRNGALLR | SEQ ID NO: 140 |
| ARGRIRNGALLRVYV | SEQ ID NO: 5 |
| RIRNGALLRVYVPRS | SEQ ID NO: 6 |
| NGALLRVYVPRSSLP | SEQ ID NO 141: |
| LLRVYVPRSSLPGFY | SEQ ID NO: 142 |
| VYVPRSSLPGFYRTS | SEQ ID NO: 143 |
| PRSSLPGFYRTSLTL | SEQ ID NO: 144 |
| SLPGFYRTSLTLAAP | SEQ ID NO: 145 |
| GFYRTSLTLAAPEAA | SEQ ID NO: 146 |
| RTSLTLAAPEAAGEV | SEQ ID NO: 147 |
| LTLAAPEAAGEVERL | SEQ ID NO: 148 |
| AAPEAAGEVERLIGH | SEQ ID NO: 149 |
| EAAGEVERLIGHPLP | SEQ ID NO: 150 |
| GEVERLIGHPLPLRL | SEQ ID NO: 151 |
| ERLIGHPLPLRLDAI | SEQ ID NO: 152 |
| IGHPLPLRLDAITGP | SEQ ID NO: 153 |
| PLPLRLDAITGPEEE | SEQ ID NO: 154 |
| LRLDAITGPEEEGGR | SEQ ID NO: 155 |
| DAITGPEEEGGRLET | SEQ ID NO: 156 |
| TGPEEEGGRLETILG | SEQ ID NO: 157 |
| EEEGGRLETILGWPL | SEQ ID NO: 158 |
| GGRLETILGWPLAER | SEQ ID NO: 159 |
| LETILGWPLAERTVV | SEQ ID NO: 160 |
| ILGWPLAERTVVIPS | SEQ ID NO: 161 |
| WPLAERTVVIPSAIP | SEQ ID NO: 162 |
| AERTVVIPSAIPTDP | SEQ ID NO: 163 |
| TVVIPSAIPTDPRNV | SEQ ID NO: 164 |
| IPSAIPTDPRNVGGD | SEQ ID NO: 165 |
| AIPTDPRNVGGDLDP | SEQ ID NO: 166 |
| TDPRNYGGDLDPSSI | SEQ ID NO: 167 |
| RNVGGDLDPSSIPDK | SEQ ID NO: 168 |
| GGDLDPSSIPDKEQA | SEQ ID NO: 169 |
| LDPSSIPDKEQAISA | SEQ ID NO: 170 |
| SSIPDKEQAISALPD | SEQ ID NO: 171 |
| PDKEQAISALPDYAS | SEQ ID NO: 172 |
| EQAISALPDYASQPG | SEQ ID NO: 173 |
| ISALPDYASQPGKPP | SEQ ID NO: 174 |
| LPDYASQPGKPPRED | SEQ ID NO: 175 |
| DYASQPGKPPREDLK | SEQ ID NO: 176 |
| MDIQMTQTTSSLSAS | SEQ ID NO: 177 |
| QMTQTTSSLSASLGD | SEQ ID NO: 178 |
| QTTSSLSASLGDRVT | SEQ ID NO: 179 |
| SSLSASLGDRVTISC | SEQ ID NO: 180 |
| SASLGDRVTISCRAS | SEQ ID NO: 181 |
| LGDRVTISCRASQDI | SEQ ID NO: 182 |
| RVTISCRASQDISNY | SEQ ID NO: 183 |
| ISCRASQDISNYLNW | SEQ ID NO: 184 |
| RASQDISNYLNWYQQ | SEQ ID NO: 185 |
| QDISNYLNWYQQKPD | SEQ ID NO: 186 |
| SNYLNWYQQKPDGTV | SEQ ID NO: 187 |
| LNWYQQKPDGTVKLL | SEQ ID NO: 188 |
| YQQKPDGTVKLLIYY | SEQ ID NO: 189 |
| KPDGTVKLLIYYTSI | SEQ ID NO: 190 |
| GTVKLLIYYTSILHS | SEQ ID NO: 191 |
| KLLIYYTSILHSGVP | SEQ ID NO: 192 |
| IYYTSILHSGVPSRF | SEQ ID NO: 193 |
| TSILHSGVPSRFSGS | SEQ ID NO: 194 |
| LHSGVPSRFSGSGSG | SEQ ID NO: 195 |
| GVPSRFSGSGSGTDY | SEQ ID NO: 196 |
| SRFSGSGSGTDYSLT | SEQ ID NO: 197 |
| SGSGSGTDYSLTISN | SEQ ID NO: 198 |
| GSGTDYSLTISNLEQ | SEQ ID NO: 199 |
| TDYSLTISNLEQEDF | SEQ ID NO: 200 |
| SLTISNLEQEDFATY | SEQ ID NO: 201 |
| ISNLEQEDFATYFCQ | SEQ ID NO: 202 |
| LEQEDFATYFCQQGN | SEQ ID NO: 203 |
| EDFATYFCQQGNTLP | SEQ ID NO: 204 |
| ATYFCQQGNTLPWTF | SEQ ID NO: 205 |
| FCQQGNTLPWTFGCG | SEQ ID NO: 206 |
| QGNTLPWTFGCGTKL | SEQ ID NO: 207 |
| TLPWTFGCGTKLEIK | SEQ ID NO: 208 |

Peptide antigen was prepared as a 2 mg/ml stock solution in DMSO. First, 0.5 microliters of the stock solution were placed in each well of the 96 well plate in which the differentiated dendritic cells were previously placed. Then, 100 microliters of the diluted CD4+T-cell solution as prepared above, were added to each well. Useful controls include diluted DMSO blanks, and tetanus toxoid positive controls.

The final concentrations in each well, at 20 microliter total volume are as follows:
2×10⁴ CD4+
2×10⁵ dendritic cells (R:S of 10:1)
5 μM peptide

### EXAMPLE 3

### I-MUNE® Assay for the Identification of Peptide T-Cell Epitopes in PE Using Human T-Cells

Once the assay reagents (i.e., cells, peptides, etc.) were prepared and distributed into the 96-well plates, the I-MUNE® assays were conducted. Controls included dendritic cells plus CD4+T-cells alone (with DMSO carrier) and with tetanus toxoid (Wyeth-Ayerst), at approximately 5 Lf/mL.

Cultures were incubated at 37° C. in 5% CO₂ for 5 days. Tritiated thymidine (NEN) was added at 0.5 microCi/well. The cultures were harvested and assessed for incorporation the next day, using the Wallac TriBeta scintillation detection system (Wallace Oy).

All tests were performed at least in duplicate. All tests reported displayed robust positive control responses to the antigen tetanus toxoid. Responses were averaged within each experiment, then normalized to the baseline response. A positive event (i.e., a proliferative response) was recorded if the response was at least 2.95 times the baseline response.

The immunogenic responses (i.e., T-cell proliferation) to the prepared peptides from PE were tallied. The overall background rate of responses to this peptide set was 5.60±3.64 for the donors tested. Using these methods various peptides of potential interest were identified, including those in Table 3, below. Peptide 75 has the sequence ARGRIRNGALLRVY (SEQ ID NO:5), while peptide 76 has the sequence RIRNGALLRVYVPRS (SEQ ID NO:6). Peptide “75-76” is a combination of peptides 75 and 76, as indicated in Table 3.

TABLE 3

Peptides of Interest in PE

| Peptide | | |
|---|---|---|
| number | Sequence | SEQ ID NO: |
| 75-76 | ARGRIRNGALLRVYVPRS | SEQ ID NO: 2 |
| (15) | RLVALYLAARLSWNQ | SEQ ID NO: 3 |
| (65) | SQDLDAIWRGFYIAG | SEQ ID NO: 4 |

Peptides #75-76, #15 and #65 were identified as being of interest, with 22.62%, 14.29% and 14.29% responders, respectively. Thus, peptide 75-76 was identified as a major (or significant) epitope, while peptides 15 and 65 were identified as minor epitopes.

As further described herein, it is contemplated that amino acid modifications in or around these peptides will provide variant PEs suitable for use as hypo-allergenic/immunogenic PEs.

### EXAMPLE 4

### HLA Association with Epitope Peptides

The HLA-DR and DQ expression of 84 donors tested in both rounds of assay testing described above were assessed using a commercially available PCR-based HLA typing kit (Bio-Synthesis). The phenotypic frequencies of individual HLA Class II DRB1 and DQB1 antigens among responders and non-responders to epitopes of interest (i.e., peptides #75, #76, #75-76, #15, and #65) were tested using a chi-squared analysis with 1 degree of freedom.

The phenotypic frequencies (presence or absence) of individual DR and DQ antigens among epitope reactive and non-reactive donors were tested using both a chi-square test (1 degree of freedom) and a Fisher's exact test. Wherever the HLA antigen was present in both reactive and non-reactive donors a relative risk (i.e., the increased or decreased likelihood of presenting a reaction conditioned on the presence of the HLA antigen) was computed. Allele frequencies among donors stratified by their reaction to the specific epitopes were also computed. In order to assess if the stimulation index differed between genotypes one-factor analyses of variance (ANOVA) were carried out using S-Plus 6 (Insightful).

Highly significant associations with both response and quantitative proliferative response to #15 and #65 and the presence of DR15 were observed. In addition, significant associations between both response and stimulation index to #75 were observed with DQ9 and to a lesser extent with DR15. The only HLA association found for #76 related to the absence of DR4 among responders. The same associations observed with #75 and #76 were seen with the combined #75-76 epitope (defined as response to either #76 or #75) although the p-values were in all instances higher (indicating weaker associations).

In sum, DR15 was found much increased among individuals that reacted to peptides #15 (2% vs 25%; p<7×10⁻⁶) and #65 (75% vs 28%; p<0.0014). For both peptides, the quantitative proliferative response (SI) was also significantly higher among DR15 carriers than among non-carriers, but only when all samples were studied. Although the trend was the same if responders and non-responders were analyzed separately, the comparison did not reach statistical significance.

DQ6, which is commonly found in the same haplotype as DR15, was also found significantly increased among responders to #15 (83% vs 40%; p<0.006). In addition, a significantly higher SI to #15 was observed among carriers of this antigen (p<0.05). The presence of this antigen was not associated, however, with response to #65 (58% in R+ vs 45% in R−).

DQ5 was decreased among responders to #15 (8% vs 39%;p<0.04) but carriers did not show a significantly lower SI than non carriers. DQ9 was significantly increased among individuals that reacted to peptide #75 (26% vs 3%; p<0.0013). The same was true, but to a lesser extent, with DR14 (26% vs 6%; p<0.015) and DR9 (16% vs 2%; p<0.011). Both carriers of DR14 and DQ9 showed a significantly higher quantitative proliferative response than non-carriers, when all donors were analyzed (p<0.001 and p<0.0001 respectively).

DQ8 was absent among responders to #75 (26% among non-responders). This difference was statistically significant with p<0.013, although the presence of this antigen did not determine a significantly lower SI.

DR4 was found at a higher frequency among non-responders than responders to #76 (8% in R+ vs 32% in R−; p<0.07). Carriers of this antigen had a lower SI than non carriers (p<0.057), although this difference was not quite statistically significant.

The same associations with HLA antigens (DR4, DR9, DQ8, DQ9, DR14) that were observed with #75 and #76 were seen with the combined #75-76 epitope (defined as response to either #76 or #75). However, in all instances, the p-values were higher and the relative risks for DR9, DQ9, and DR14 were all lower, indicating weaker associations between these HLA antigens and response to the combined peptide. The results obtained in these experiments indicate that HLA antigens appear to modulate the proliferative response to all the epitopes studied in PE38. The strongest of these are between DR15 and peptides #15 and #65 and DQ9 and peptide #75.

Thus, the magnitude of the proliferative response to an individual peptide in responders and non-responders expressing epitope-associated HLA alleles were analyzed. An “individual responder to the peptide” is defined by a stimulation index of greater than 2.95. It is contemplated that the proliferative responses in donors who express an epitope associated with HLA alleles are higher than in peptide responders who do not express the associated allele.

From the above, it is clear that the present invention provides methods and compositions for the identification of T-cell epitopes in wild-type PE. Once antigenic epitopes are identified, the epitopes are modified as desired, and the peptide sequences of the modified epitopes incorporated into a wild-type PE, so that the modified sequence is no longer capable of initiating the CD4+T-cell response or wherein the CD4+T-cell response is significantly reduced in comparison to the wild-type parent. In particular, the present invention provides means, including methods and compositions suitable for reducing the immunogenicity of PE.

### EXAMPLE 5

### Critical Residue Testing

In this Example, critical residue testing experiments for variants of peptides #75-76, #15, and #65 are described. In these experiments, alanine scans are performed for each peptide in order to produce variants of each of the parent peptides (i.e., peptides #15, #75-76 and #65). These variant peptides are synthesized using the multi-pin synthesis technique known in the art (See e.g., Maeji et al., J. Immunol. Meth., 134:23-33 [1990]).

The assay is performed as described in Example 3, utilizing the variant peptides on a set of donor samples. Proliferative responses are collated, and the results analyzed.

### EXAMPLE 6

### Epitope Modifications

As indicated above, specific amino acid substitutions in various peptides of interest are tested in the I-MUNE® assay (see above) on an additional set of donors along with the alanine scan mutagenized peptides. These peptides are then tested as 15-mer peptides offset by 3 amino acids across the peptide sequence of PE that encompasses epitope ##75-76, #15 and #65. These tests are performed in order to ensure that the amino acid variants did not introduce a de novo CD4+T-cell epitope in another frame.

### EXAMPLE 7

### PBMC Proliferation Assay

In this Example, experiments conducted to assess the ability of PE and epitope-modified PE to stimulate PBMCs are described. All of the proteins are purified to approximately 2 mg/ml.

The blood samples used in these experiments are the same as described above (i.e., before Example 1). The PBMCs are separated using Lymphoprep, as known in the art. The PBMCs are washed in PBS and counted using a Cell Dyn® 3700 blood analyzer (Abbott). The cell numbers and differentials are recorded. The PBMCs are resuspended to 4×10⁶ cells/ml, in a solution of heat-inactivated human AB serum, RPMI 1640, pen/strep, glutamine, and 2-ME. Then, 2 mls per well are plated into 24-well plates. Two wells are used as no-enzyme controls. Then, the unmodified PE and modified PEs are added to the wells at concentrations of 10 ug/ml, 20 ug/ml, and 40 ug/ml. The plates are incubated at 37° C., in a 5% CO₂, humidified atmosphere for 6-7 days. On the day of harvest, the cells in each well are mixed and resuspended in the wells. Then, 8 aliquots of 100 ul from each well are transferred to a 96-well microtiter plate. To these wells, 0.25 uCi of tritiated thymidine are added. These plates are incubated for 6 hours, the cells harvested and counted. For analysis, the data for the eight replicates from each well are averaged. For the controls, the two wells are sampled to provide a total of 32 replicates. Each set of eight control wells are averaged, and the four average values used to calculate a coefficient of variation (CV) for each donor. SI values are calculated by dividing the average for each set of eight wells for each sample by the average CPM for the control well. The data are analyzed by creating a dataset representing the highest SI value achieved for each donor and each enzyme.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described modes for carrying out the invention which that are obvious to those skilled in molecular biology, immunology, formulations, and/or related fields are intended to be within the scope of the present invention.

1. A Pseudomonas exotoxin A (PE) variant comprising at least one mutation compared with a parent PE, wherein at least one said mutation is within at least one peptide sequence corresponding to one or more of SEQ ID NOs 2, 3, 4, 5, and 6 within the parent PE;
with the proviso that where variant comprises an amino acid substitution at the arginine residue within SEQ ID NOs 2 and 6 corresponding to position 224 in SEQ ID NO: 1, there is at least one additional mutation within at least one peptide sequence corresponding to one or more of SEQ ID NOs 2, 3, 4, 5, and 6 within the parent PE.

2. A PE variant according to claim 1 wherein the at least one mutation is made within the peptide sequence corresponding to SEQ ID NO: 2.

3. A PE variant according to claim 1 wherein the parent PE is wild-type PE.

4. A PE variant according to claim 1 wherein the parent PE is PE38.

5. A PE variant according to claim 3 wherein the variant comprises a mutation at the arginine residue corresponding to position 224 in SEQ ID NO: 1.

6. A PE variant according to claim 4 wherein the variant comprises a mutation at the arginine residue corresponding to position 224 in SEQ ID NO: 1.

7. A PE variant according to claim 1 wherein the variant comprises the substitution at said arginine residue corresponding to position 224 in SEQ ID NO: 1, and wherein the substitution is to alanine.

8. A PE variant according to claim 1 which has reduced immunogenicity compared with the parent PE.

9. A PE variant according to claim 1 conjugated to an antibody molecule.

10. A PE variant according to claim 9 wherein the antibody molecule is selected from the group consisting of anti-CD22, anti-mesothelin, anti-CD25 and anti-Lewis Y antibody molecules.

11. A PE variant according to claim 10 wherein the antibody molecule is an anti-CD22 antibody molecule.

12. A PE variant according to claim 1 conjugated to a cancer-targeting molecule.

13. (canceled)

14. A PE variant according to claim 9 wherein the PE variant is conjugated to the antibody molecule via a peptide bond as part of a fusion protein comprising the antibody molecule.

15. An isolated nucleic acid encoding a PE variant according to claim 1.

16. An expression vector comprising nucleic acid encoding a PE variant according to claim 1.

17. A host cell transformed with an expression vector according to claim 16.

18. A method of producing a PE variant comprising culturing a host cell according to claim 17 and purifying the variant expressed from the encoding nucleic acid.

19. (canceled)

20. A method of producing a PE variant according to claim 1 comprising mutating a nucleic acid sequence encoding the parent PE to provide nucleic acid encoding the PE variant, transforming a host cell with an expression vector comprising the mutated nucleic acid encoding the PE variant, and purifying the PE variant expressed from the encoding nucleic acid.

21. (canceled)

22. (canceled)

23. A method according to claim 20 further comprising formulating the variant into a composition comprising pharmaceutically acceptable components.

24. An isolated peptide consisting of the amino acid sequence of any of SEQ ID NOs 2, 3, 4, 5, and 6.
